(19) United States
(12) Patent Application Publication
(10) Pub. No.: US 2014/0163082 A1
(43) Pub. Date: Jun. 12, 2014

(54) PYRAZOLYL COMPOUNDS FOR USE IN REVERSING REACTIVE GLIOSIS

(71) Applicants: Futwan AL-Mohanna, Riyadh (SA); Michael Deniro, Riyadh (SA)

(72) Inventors: Futwan AL-Mohanna, Riyadh (SA); Michael Deniro, Riyadh (SA)

(73) Assignees: KING KHALID EYE SPECIALIST HOSPITAL, RIYADH (SA); TERRAMARK MARKENCREATION GMBH, BREMEN (DE)

(21) Appl. No.: 14/008,559

(22) PCT Filed: May 6, 2011
(86) PCT No.: PCT/EP11/02276

§ 371 Date: Jan. 4, 2014

(51) Int. Cl. C07D 405/04

(52) U.S. Cl. CPC C07D 405/04
USPC 514/403

(57) ABSTRACT

The present invention refers to a compound which is a pyrazolyl compound for use as a medicament in a neurodegenerative disease or disorder, preferably reactive gliosis. Also, a pyrazolyl compound that is 3-(5′-hydroxymethyl-2′-furyl)-1-benzylindazole (YC-1) for use as a medicament in a disease or disorder, preferably for the treatment, reversal or the attenuation of reactive gliosis, and/or reactive gliosis directly or indirectly associated with an eye disease, disease of the retina, or retinal disorder. Further, the invention discloses pharmaceutical compositions thereof and a method for treating a neurodegenerative disorder, preferably reactive gliosis, comprising administering to a subject in need thereof an effective amount of such compound or pharmaceutical composition.

The present invention refers to a compound which is a pyrazolyl compound for use as a medicament for the treatment of reactive gliosis directly or indirectly associated with eye diseases, diseases of the retina, retinal disorders, which include but not limited to: retinopathy, ischemic retinopathy, hypertensive retinopathy, retinal neovascularization, macular degeneration [age-related macular degeneration (AMD)], Meckel syndrome, autosomal recessive inheritance diseases, Bardet-Biedl syndrome, retinal vessel occlusions [blockage of central retinal arteries and veins][branch and central retinal vein occlusions] and retinal artery occlusion, cytomegalovirus (CMV) retinitis, diabetic retinopathy [retinopathy in diabetes], diabetic eye problems, epi-retinal membrane (or cellophane or macular pucker), flashin lights, eye floaters, flashes and posterior vitreous detachments, macular edema (CME), macular holes, macular translocation, cancers affecting the retina, melanoma, retinoblastoma (PDQ), retinal tear and retinal detachment, retinal detachment repair, proliferative vitreoretinopathy (PVR), photodynamic therapy (PDT), subretinal neovascular membranes and surgery (AMD, OHS, idiopathic, myopia, PXE, etc.), vitrectomy, usher syndrome, retinoschisis, retinitis pigmentosa (RP), retinal tear, Bietti's crystalline dystrophy, choroideremia, retinopathy of prematurity (ROP), Behcet's disease, central serous choroidopathy, amaurosis fugax, Leber congenital amaurosis, juvenile retinoschisis, Refsum disease, neuropathy, ataxia, and retinitis pigmentosa, Leber congenital amaurosis, familial exudative vitreoretinopathy, and choroideremia. Also, a pyrazolyl compound that is 3-(5′-hydroxymethyl-2′-furyl)-1-benzylindazole (YC-1) for use as a medicament for the treatment of reactive gliosis directly or indirectly associated with eye diseases, diseases of the retina, or retinal disorders. Further, the invention discloses pharmaceutical compositions thereof and a method for treating a, reactive gliosis directly or indirectly associated with eye diseases, diseases of the retina, retinal disorders, comprising administering to a subject in need thereof an effective amount of such compound or pharmaceutical composition.

### BACKGROUND OF THE INVENTION

Diabetic retinopathy [DR] is a leading cause of visual disturbance in adults and is the leading cause of blindness in Americans between the ages of 20 and 74 years [48]. DR has been regarded as a retinal microvascular disease, which develops in two stages: an early, nonproliferative stage, and a later, proliferative stage. In the early non-proliferative stage, retinal vascular permeability can increase even before the appearance of clinical retinopathy [59]. Currently, this stage is diagnosed by dilation of retinal veins, retinal microaneurysmas, intraretinal microvascular abnormalities [which include intraretinal new vessels], areas of capillary nonperfusion, retinal hemorrhages, cotton wool spots [infarctions within the nerve fiber layer [NFL]], edema, and exudates. All these signs indicate regional failure of the retinal microvascular circulation, which presumably results in ischemia. On the other hand, proliferative DR [PDR] is diagnosed based on the ischemia-induced formation of new blood vessels on the surface of the retina. New vessels can extend into the vitreous cavity of the eye and can hemorrhage into the vitreous, resulting in visual loss [7]. They also can cause tractional retinal detachments from the accompanying contractile fibrous tissue. Furthermore, during this stage, over-proliferation of capillary endothelial cells [EC] results in retinal neovascularisation [NV], abnormal formation of new vessels in the retina and in the vitreous, leading to PDR [68]. As a consequence retinal edema may ensue [15]. Retinal edema involves the breakdown of the blood-retinal barrier, with leakage of plasma from small blood vessels.

Platelet-derived growth factor-B [PDGF-B] is secreted by endothelial cells. PDGF-B is both chemotactic and mitogenic to vascular endothelial cells in vitro [56] and may also have angiogenic effects in vivo [40]. Increased expression of PDGF-B in the retina causes severe proliferative retinopathy and retinal detachment similar to advanced stages of PDR [64]. PDGF-B promotes the recruitment, proliferation and survival of pericytes; recruits glial cells and retinal pigment epithelial [RPE] cells [9], which promotes scarring, a complication of ocular NV that is the major cause of permanent loss of vision.

Nitric oxide [NO] is an important signaling pathway that mediates a variety of essential physiological processes, including neurotransmission, vasodilatation, and host cell defense [44]. NO is generated from L-arginine by the catalytic reaction of three different isoforms of nitric oxide synthase [NOS]. Neuronal and endothelial isoforms are constitutively expressed [cNOS] and upregulated by intracellular free calcium. At low concentrations NO regulates vessel tone; whereas at high concentrations NO mediates tissue damage [47]. In addition, NO directly contributes to tissue damage by combining with superoxide to form peroxynitrite, a highly reactive species that produces lipid peroxidation, mitochondrial and DNA damage. Moreover, inducible NOS [iNOS] is expressed at the transcriptional level by macrophages, neutrophils and a number of other cells in response to inflammatory stimuli such as LPS and cytokines [47]. iNOS is expressed in RPE, ciliary epithelial cells, Müller cells, retinal parenchyma, choroid vasculature and pericytes [45]. In addition, iNOS is independent of calcium, and generates large amounts of NO [nanomolar concentrations] over extended periods [hours to days]. It is believed that the relatively smaller amount of NO generated by eNOS and nNOS are involved in physiological functions, whereas the comparatively larger amount of NO generated by iNOS is involved in the pathophysiology of a number of inflammatory diseases [47]. Upregulation of iNOS level has been found in retinas of experimental diabetic rodents and human patients in most studies [19]. Furthermore, NO has been shown to have pro-angiogenic or anti-angiogenic effects depending upon the physiological or the pathological setting. In transgenic mice with increased expression of VEGF in photoreceptors, deficiency of any of the three isoforms caused a significant decrease in subretinal NV [10]. In mice with laser-induced rupture of Bruch's membrane, deficiency of iNOS or nNOS, but not eNOS, caused a significant decrease in choroidal NV. In the oxygen induced retinopathy [OIR] mouse model, deficiency of eNOS, but not iNOS or nNOS, caused a significant decrease in retinal NV and decreased expression of VEGF. These data suggest that NO contributes to both retinal and choroidal NV, but that different isoforms of NOS are involved. Several studies demonstrated that NO plays a critical role in VEGF-induced vascular hyperpermeability [41] and angiogenesis [49]. Furthermore, VEGF was shown to induce the expression of iNOS [39] and stimulate production of NO [72].

The retina contains two types of macroglial cells. The most abundant are the Müller cells, which project from the retinal ganglion cell layer [GCL] to the photoreceptors, whereas the astrocytes, which originate in the optic nerve and migrate into the retina during development [71] reside as a single layer adjacent to the inner limiting membrane.

Müller cells are the principal glial cells of the neural retina, and play a wealth of crucial roles in supporting neuronal function [4]. In response to virtually every pathological alteration of the ischemia, photic damage, retinal trauma, retinal detachment, glaucoma, DR, and age-related macular degeneration, Müller cells become reactivated [4]. Müller cells protect neurons after retinal injury, via release of neurotrophic factors and free radical scavengers, glutamate uptake, and facilitation of NV [4]. Hence, the close association between neurons and glia suggests that gene expression in these cell types is likely to be influenced by mutual interactions. Several lines of evidence indicate that glia influence the growth, migration and differentiation of neurons. Glial cells provide structural and metabolic support for retinal neurons and blood vessels, and the cells become reactive in certain injury states [35].

It has been shown in animal studies that iNOS is localized at the GCL, at the inner nuclear layer [INL], and at the outer nuclear layer [ONL] in the retinas of diabetic rats. iNOS immunoreactivity was observed in the retina in other ischemic retinopathies as well. Retinal ischemia induced by common carotid artery occlusion in rats induced iNOS expression in Müller cells and retinal ganglion cells. In a murine model of OIR, iNOS mRNA was expressed in ischemic retina [16]: Ischemia has been shown to induce the expression of iNOS [62] and VEGF [46] in the retina. Yet, other studies demonstrated that VEGF induced the expression of iNOS in human EC [39] Recently, several studies demonstrated expression of VEGF receptors in retinal ganglion cells, INL, and Müller cells [51]. Induction of iNOS through VEGF stimulates production of NO from rabbit and human EC through activation of tyrosine kinases and an increase in intracellular calcium [72]. Several studies demonstrated that NO plays a critical role in VEGF-induced vascular hyperpermeability and angiogenesis. Previous studies demonstrated that an NOS inhibitor blocked VEGF-induced vascular hyperpermeability in all ocular and non-ocular tissues. Since the expression of iNOS protein was mainly localized at the level of the retinal vessels, it is possible that the overproduction of NO by the iNOS isoform contributes to blood-retinal barrier breakdown in the ischemic retinas of OIR mouse model. Since NOS inhibitors block the VEGF-induced proliferation, the mitogenic action of VEGF on EC is likely to be NO mediated [66]. In a murine model of OIR, iNOS expression was found to inhibit angiogenesis locally in the avascular retina mediated by a downregulation of VEGF R2 and to promote intravitreal NV [62]. iNOS inhibitors enhanced angiogenesis in the ischemic retina and inhibited pathological intravitreal NV. In addition, pathological intravitreal NV was significantly reduced in iNOS knockout mice [62]. In addition, data have demonstrated that that oxygen-induced retinal vaso-obliteration was significantly reduced by an NOS inhibitor, strongly supporting a putative role for NO in the retinal vaso-obliterative process [5].

Several studies suggested that glial reactivity and altered glial metabolism are early pathological events in the retina during diabetes [58]. The most constant manifestation of reactivity is the increase in immunoreactivity for the intermediate filament protein glial fibrillary acidic protein [GFAP][43]. GFAP is mainly expressed in astrocytes for which it constitutes a selective marker. Previous reports have demonstrated that upregulation of astrocytic intermediate filaments is a crucial step and a hallmark of reactive gliosis [RG][54].

RG is one of the pathophysiological features of retinal damage. RG includes morphological, biochemical, and physiological changes of Müller cells; these alterations vary with type and severity of insult. Under stress, Müller cells exhibit three crucial nonspecific gliotic responses, which are considered as “hallmarks of glial cell activation”, these are: [i] cellular hypertrophy due to alterations in intermediate filament [10], [ii] cellular proliferation [8], and [iii] the upregulation of the intermediate filament [IF] system [known also as nanofilament system] composed of GFAP, vimentin, nestin and synemin [32, 55]. There are other gliotic characteristics such; targeted cellular migration [69], changes in ion transport properties [57], and secretion of signaling molecules such as VEGF [1]. Like other glial cells of the CNS, Müller cells undergo RG following acute retinal injury or chronic neuronal stress [73]. The overexpression of GFAP is the most sensitive non-specific response to retinal disease and injury, and it may be considered as the hallmark of “retinal stress”, i.e. as a universal early cellular marker for retinal injury and Müller cell activation [4]. Furthermore, GFAP, which is located primarily to Müller cells, has specific immunoreactivities that occur in all retinal eccentricities. The vertebrate retina contains a specialized type of glia, the Müller glia, not found elsewhere in the CNS. Müller cell gliosis is characterized by proliferation [61], and changes in cell shape due to alterations in intermediate filament [60]. Successful inhibition of GFAP using antisense oligonucleotides has also been reported by several groups [71, 72, 40]. Ostensibly, gliosis is important for the protection and repair of retinal neurons, yet some pathologies such as DR may be exacerbated by RG properties [55, 1].

In general, neuronal loss in the retina due to various eye and retinal diseases that are associated directly or indirectly with Reactive Gliosis. Such diseases include but are not limited to: retinopathy, ischemic retinopathy, hypertensive retinopathy, retinal neovascularization, macular degeneration [age-related macular degeneration (AMD)], Meckel syndrome, autosomal recessive inheritance diseases, Bardet-Biedl syndrome, retinal vessel occlusions [blockage of central retinal arteries and veins] [branch and central retinal vein occlusions] and retinal artery occlusion, cytomegalovirus (CMV) retinitis, diabetic retinopathy [retinopathy in diabetes], diabetic eye problems, epi-retinal membrane (or cellophane or macular pucker), flashin lights, eye floaters, flashes and posterior vitreous detachments, macular edema (CME), macular holes, macular translocation, cancers affecting the retina, melanoma, retinoblastoma (PDQ), retinal tear and retinal detachment, retinal detachment repair, proliferative vitreoretinopathy (PVR), photodynamic therapy (PDT), subretinal neovascular membranes and surgery (AMD, OHS, idiopathic, myopia, PXE, etc.), vitrectomy, Usher syndrome, retinoschisis, retinitis pigmentosa (RP), retinal tear, Bietti's crystalline dystrophy, choroideremia, retinopathy of prematurity (ROP), Behcet's disease, central serous choroidopathy, amaurosis fugax, Leber congenital amaurosis, juvenile retinoschisis, Refsum disease, neuropathy, ataxia, and retinitis pigmentosa, Leber congenital amaurosis, familial exudative vitreoretinopathy, and choroideremia.

Few medicaments to treat reactive gliosis in the retinal or eye diseases are currently known. Thus, there is a strong need for novel effective substances and formulations for use as a medicament for the treatment, reversal or the attenuation of reactive gliosis and reactive gliosis associated with one of the aforementioned diseases.

So far, a method for treating a neurodegenerative disease, comprising administrating a pyrazolyl compound like 1-benzyl-3-(5′-hydroxymethyl-2′-furyl)-indazole or 1-benzyl-3-(5′-methoxymethyl-2′-furyl)-indazole has been previously described in US 2004/0077702. In contrast, reactive gliosis is an acute disease, while a neurodegenerative disease is a chronic disease. Reactive gliosis may lead to a neurodegenerative disease, however, this is not necessarily the case.

The object of the invention is thus to provide an alternative substance for use as a medicament for the treatment, reversal or the attenuation of reactive gliosis and reactive gliosis associated with one of the aforementioned diseases.

### SUMMARY OF THE INVENTION

The object of the present invention is solved by the subject-matter as defined in the attached claims. The object of the invention is solved by a pyrazolyl compound comprising the chemical structure of formula I

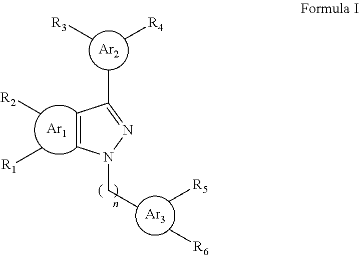

wherein Ar₁ forms an aromatic or heteroaromatic ring, or is phenyl, wherein the aromatic or heteroaromatic ring is optionally substituted; Ar₂ is selected from furyl, phenyl, alkyl, aryl, or heterocyclyl; wherein the alkyl, aryl, or heterocyclyl is optionally substituted; Ar₃ is phenyl or substituted phenyl; n is 1 to 10; or any enantiomer, racemic form or mixture, prodrug, or analog of the substance, for use as a medicament for the treatment, reversal or the attenuation of reactive gliosis, and/or reactive gliosis directly or indirectly associated with eye diseases, diseases of the retina, or retinal disorders.

In a preferred embodiment, the object of the invention is solved by a pyrazolyl compound comprising the chemical of structure formula I, wherein

each of Ar₁ and Ar₃ is phenyl;

Ar₂ is furyl or phenyl;

one of R₁, and R₂, is H, and the other is H, C₁-C₆ alkyl or halogen; or both R₁ and R₂ are H;

one of R₃ and R₄ is H, and the other is CH₂OH;

each of R₅ and R₆ is H; n is 1 to 10

or any enantiomer, racemic form or mixture, prodrug, or analog of the substance, for use as a medicament for the treatment, reversal or the attenuation of reactive gliosis and/or reactive gliosis directly or indirectly associated with eye diseases, diseases of the retina, or retinal disorders.

In one embodiment, the compound is the compound as described above, wherein Ar₂ is 5′-furyl. In a further embodiment, R₃ is substituted at position 2 of furyl and R₃ is CH₂OH and R₄ is H. In a further embodiment, R₁ and R₂, are substituted at positions 4 and 5 of phenyl and R₁ is H, and R₂ is CH₃. In yet a further embodiment, R₁ is H, and R₂ is F.

Preferably, the object is solved by the compound which is 3-(5′-hydroxymethyl-2′-furyl)-1-benzylindazole (YC-1) and has the chemical structure of Formula II:

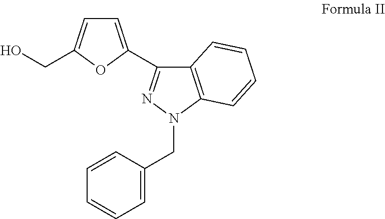 [SMILES: OCc1ccc(-c2nn(Cc3ccccc3)c3ccccc23)o1]

or any enantiomer, racemic form or mixture, prodrug, or analog of the substance.

In another preferred embodiment, the object is solved by the compound, which is 1-benzyl-3-(5-methyl-furan-2-yl)-1H-indazole, 1-benzyl-3-(5-methoxymethyl-furan-2-yl)-1H-indazole, or 1-benzyl-3-(5′-methoxymethyl-2′-furyl)-indazole, or any enantiomer, racemic form or mixture, prodrug, or analog of the substance.

The compound of the invention may also be for use in reduction of PDGF-B, GFAP and iNOS expression and protein levels, in vivo and in vitro; reduction of filopodial length and number in endothelial tip cells; inhibition of ischemic pathologic neovascular response; promotion of the physiological retinal microvascular repair and intra-retinal revascularization [RV] of the avascular retina.

In yet another preferred embodiment, the compound as described above is for use as a medicament for the treatment, reversal or the attenuation of gliosis, astrogliosis, reactive gliosis, preferably reactive gliosis, more preferably reactive gliosis that is directly or indirectly associated with retinal or eye diseases.

In yet another preferred embodiment, the compound as described above is for use as a medicament for the treatment, reversal or the attenuation of reactive gliosis associated with retinopathy, ischemic retinopathy, hypertensive retinopathy, retinal neovascularization, macular degeneration [age-related macular degeneration (AMD)], Meckel syndrome, autosomal recessive inheritance diseases, Bardet-Biedl syndrome, retinal vessel occlusions [blockage of central retinal arteries and veins] [branch and central retinal vein occlusions] and retinal artery occlusion, cytomegalovirus (CMV) retinitis, diabetic retinopathy [retinopathy in diabetes], diabetic eye problems, epi-retinal membrane (or cellophane or macular pucker), flashin lights, eye floaters, flashes and posterior vitreous detachments, macular edema (CME), macular holes, macular translocation, cancers affecting the retina, melanoma, retinoblastoma (PDQ), retinal tear and retinal detachment, retinal detachment repair, proliferative Vitreoretinopathy (PVR), photodynamic therapy (PDT), subretinal neovascular membranes and surgery (AMD, OHS, idiopathic, myopia, PXE, etc.), vitrectomy, usher syndrome, retinoschisis, retinitis pigmentosa (RP), retinal tear, Bietti's crystalline dystrophy, choroideremia, retinopathy of prematurity (ROP), Behcet's disease, central serous choroidopathy, amaurosis fugax, Leber congenital amaurosis, juvenile retinoschisis, refsum disease, neuropathy, ataxia, retinitis pigmentosa, Leber congenital amaurosis, familial exudative vitreoretinopathy, and/or choroideremia.

The object of the invention is also solved by a pharmaceutical composition comprising the compound as described above and a pharmaceutical acceptable carrier for use as a medicament for the treatment, reversal, or attenuation of an acute neuronal disease or disorder, preferably reactive gliosis, directly or indirectly associated with eye diseases, diseases of the retina, or retinal disorders. In a preferred embodiment, the acute disorder is reactive gliosis, preferably reactive gliosis associated with one of the aforementioned diseases.

The object of the invention is also solved by a pharmaceutical composition for use in reduction of PDGF-B, GFAP and iNOS expression and protein levels, in vivo and in vitro; reduction of filopodial length and number in endothelial tip cells; inhibition of ischemic pathologic neovascular response; promotion of the physiological retinal microvascular repair and intra-retinal revascularization [RV] of the avascular retina.

In yet another embodiment, the pharmaceutical composition as described above is for use as a medicament for the treatment, reversal, or attenuation of a disease or disorder that is reactive gliosis, reactive gliosis directly or indirectly associated with eye diseases, diseases of the retina, retinal disorders, preferably reactive gliosis associated with one of the aforementioned diseases, or reactive gliosis associated with retinopathy, ischemic retinopathy, hypertensive retinopathy, retinal neovascularization, macular degeneration [age-related macular degeneration (AMD)], Meckel syndrome, autosomal recessive inheritance diseases, Bardet-Biedl syndrome, retinal vessel occlusions [blockage of central retinal arteries and veins] [branch and central retinal vein occlusions] and retinal artery occlusion, cytomegalovirus (CMV) retinitis, diabetic retinopathy [retinopathy in diabetes], diabetic eye problems, epi-retinal membrane (or cellophane or macular pucker), flashin lights, eye floaters, flashes and posterior vitreous detachments, macular edema (CME), macular holes, macular translocation, cancers affecting the retina, melanoma, retinoblastoma (PDQ), retinal tear and retinal detachment, retinal detachment repair, proliferative vitreoretinopathy (PVR), photodynamic therapy (PDT), subretinal neovascular membranes and surgery (AMD, OHS, idiopathic, myopia, PXE, etc.), vitrectomy, Usher syndrome, retinoschisis, retinitis pigmentosa (RP), retinal tear, Bietti's crystalline dystrophy, choroideremia, retinopathy of prematurity (ROP), Behcet's disease, central serous choroidopathy, amaurosis fugax, Leber congenital amaurosis, juvenile retinoschisis, refsum disease, neuropathy, ataxia, retinitis pigmentosa, Leber congenital amaurosis, familial exudative vitreoretinopathy, and/or choroideremia.

In another preferred embodiment, the dosage form of the pharmaceutical composition is a tablet, lozenge, pill, dragee, capsule, liquid, gel, syrup, slurry, suspension, solution or emulsion. In another preferred embodiment, the pharmaceutical composition is for oral, rectal, transmucosal, transdermal, intestinal, parenteral, intramuscular, intrathecal, direct intraventricular, intravenous, intraperitoneal, intranasal, or intraocular, or subcutaneous administration.

The object of the invention is further solved by a method for treating an acute disorder and the treatment, reversal or the attenuation of reactive gliosis and reactive gliosis associated with one of the aforementioned diseases, comprising administering to a subject in need thereof an effective amount of a therapeutic agent comprising a compound as described above or a pharmaceutical composition as described above. Preferably, the method comprises the administration of 3-(5′-hydroxymethyl-2′-furyl)-1-benzylindazole and the acute disease or disorder is one of the diseases or disorders as mentioned above, reactive gliosis, preferably reactive gliosis associated with one of the aforementioned diseases.

### DESCRIPTION OF THE PREFERRED EMBODIMENTS OF THE INVENTION

The invention delves into the reversal of reactive gliosis directly or indirectly associated with eye diseases, diseases of the retina, retinal disorders, in particular of RG, which will consequentially rescue the Müller glial cells and the neurosensory retina during ischemic retinopathy. The possibility of interfering with this detrimental cycle by pharmacologically reversing RG has been proposed as a novel rationale to blunt neuronal damage and consequently slow the course of disease. The substances and pharmaceutical compositions of the invention are able to reverse retinal tissue damage by administration of YC-1, as this small nontoxic molecule is one of the few molecules that could exert its protective effects and reverse RG cascade and therefore rescue the neurosensory retina from a paramount damage. It is conceivable that the reversal of RG results is a niche that is more permissive to neurogenesis and the survival of newly formed neurons after ischemia. Data have demonstrated that during continuous hyperoxia, vitreous neovascularization was blocked, whereas retinal revascularization was simultaneously accelerated [27]. The same has suggested that sustained hyperoxia effectively preserved astrocytes in the central retina in spite of extensive damage to the capillary networks and prevented the reactive expression of GFAP in the Müller glia. In the Examples, the OIR mouse model is utilized because it constitutes several clinical manifestations that are analogous to the retinopathy of maturity [ROP], and mimics the important aspects of PDR; the most common ischemic retinopathy in patients [37]. Since YC-1 appeared to induce physiological RV in the OIR model, and a significant segment of this process seems to occur in the INL, OPL, ONL, and OLM retinal cell layers.

YC-1, [345′-hydroxymethyl-2′ furyl]-1-benzyl indazole], is a small molecule that inhibits cGMP breakdown and potentiates NO-induced soluble guanylyl cyclase [sGC] stimulation [38, 23]. Previous data have revealed that YC-1 suppressed retinal new vessel growth and formation in human retinal microvascular EC, and retinal explants. Furthermore, it can be demonstrated that YC-1 down-regulates HIF-1α, HIF-2α, VEGF, EPO, ET-1, and MMP-9 protein levels in the human retinal microvascular EC [16, 17, 18]. The primary objectives of the Examples of the invention disclosed herein is to determine; [i] whether ischemic exposure in the OIR mouse model induces RG, and the upregulation of GFAP neurosensory marker; [ii] whether YC-1 can rescue the neurosensory retina against the influence of ischemic exposure. The current study disclosed by the description of the invention investigates the efficacy of YC-1 in modulating iNOS expression as a therapeutic modality to target retinal NV in vivo, and examines the therapeutic potentials of utilizing YC-1 as a HIF-1 and an iNOS inhibitor. YC-1 may directly sculpt the microenvironment within the vascular plexus by exerting notable in vivo pleiotropic vascular effects, which alludes to its potential use as a promising therapeutic agent in clinical applications.

U.S. Pat. No. 7,226,941 discloses therapeutic use of YC-1 for proliferative disorders, such as angiogenesis, but is silent on its effect on neurodegenerative diseases. Disclosed herein is a fused pyrazolyl compound for use as a medicament for the treatment, reversal, or attenuation of the acute stage of reactive gliosis disorder and disease, astrogliosis, or reactive gliosis. In a preferred embodiment, the reactive gliosis disorder is astrogliosis or reactive gliosis. Most preferably, the disorder is reactive gliosis.

The pyrazolyl compound of the invention comprises a chemical structure as depicted in Formula I:

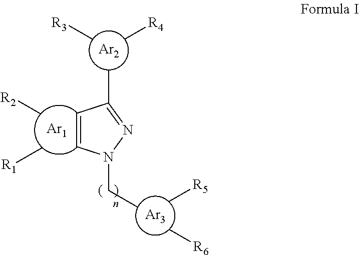

wherein each of Ar₁, and Ar₃ is phenyl; Ar₂ is furyl or phenyl; one of R₁, and R₂, is H, and the other is C₁-C₆ alkyl or halogen; or both R₁, and R₂ are H; one of R₃ and R₄ is H, and the other is CH₂OH; each of R₅ and R₆ is H; and n is 1 to 10.

Preferably, Ar₂ is 5′-furyl. In another embodiment of the invention, Ar₂ is 5′-furyl, R₃ is substituted at position 2 of furyl and is CH₂OH and R₄ is H. In yet another embodiment of the invention, Ar₂ is 5′-furyl, R₁ and R₂ are substituted at positions 4 and 5 of phenyl and R₁ is H, and R₂ is CH₃ or F.

In a most preferred embodiment, the pyrazolyl compound is 3-(5′-hydroxymethyl-2′-furyl)-1-benzylindazole (YC-1) and has the chemical structure of Formula II:

Preferably, 3-(5′-

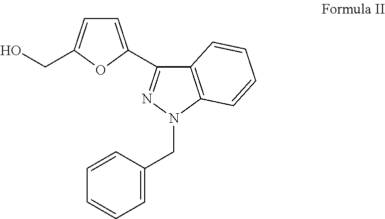 [SMILES: OCc1ccc(-c2nn(Cc3ccccc3)c3ccccc23)o1]

hydroxymethyl-2′-furyl)-1-benzylindazole (YC-1) is for use as a medicament for the treatment, reversal, or attenuation of reactive gliosis, preferably reactive gliosis directly or indirectly associated with eye diseases, diseases of the retina, or retinal disorders.

The immunocytochemical characterization of GFAP showed low expression levels under normoxic conditions, in vitro and in vivo, which is in concordance with what's been previously reported [73]. In addition, the immunocytochemical/immunohistochemical analyses have indicated that upon exposure to hypoxic/ischemic conditions, GFAP levels were increased both at the message and the protein levels. This is in concordance with what's been previously reported by other studies, which indicted that hypoxic subculture of Müller cells cause a dramatic upregulation of GFAP in vitro [11]. Previous investigations [34, 36] have both indicated that retinal ischemia/hypoxia causes an increase in gene and protein expression of GFAP in Müller cells within 1-3 h in which Müller cells exhibited immunopositive staining for GFAP within their inner processes [up to the INL]. Normally, astrocytes are the only cells in the retina that express GFAP, but after various insults such as retinal detachment, ischemia, light damage, or genetic photoreceptor degeneration, Müller glia become GFAP-positive [20, 22].

The disclosure of this invention reveals that in the non-ischemic retinas, weak low expression levels of GFAP immunoreactivity was primarily confined to the NFL and the GCL, while Müller cell processes were not stained. Whereas in the ischemic retinas, GFAP was significantly up-regulated the in the IPL, INL, and ONL. Additionally, GFAP induction in Müller cell processes end feet was clearly visible, and it was overly expressed due to the damage to the retina by ischemia after vascular pruning, which indicates glial reactivity. Therefore, considering that the dramatic induction of GFAP expression is characteristic of many ocular and neuropathological disorders that accompany excessive RG, finding a mechanism in which GFAP is upregulated in retinal RG could be a useful target for preventing or treating these kinds of ocular disorders.

As an example for the invention, YC-1 is a small molecule that inhibits cGMP breakdown and potentiates NO-induced soluble guanylyl cyclase [sGC] stimulation [23, 38]. YC-1 is a HIF-1 inhibitor, which display antiangiogenic activities in vitro and in vivo [16, 18]. YC-1 possesses novel pleiotropic effects pertaining the making, toning, maintaining the structural and functional integrities of blood vessels. The invention, in particular YC-1, represses GFAP expression at the message and the protein levels, in the ischemic retinas and in glial cells, and thus leads to a reversal of RG. No other reports to date have demonstrated the ability of small anti-angiogenic molecules to reverse RG and rescue the neurosensory retina.

Previous studies have demonstrated that levels of PDGF-B were increased after ischemic injury [28]. Under hypoxic conditions PDGF-B is expressed at significantly higher levels compared to normoxic retinas (FIG. 5G, 6 (PDGF slides), 8C (PDGF bands for Müller and glia cells)). YC-1-treated retinas exhibited a significant inhibition of PDGF-B mRNA and protein levels in the ischemic retinas, when compared to non-treated and DMSO-treated oxygen-injured retinas. It is noteworthy that other studies have attributed the retinal production of PDGF-B to the ganglion cells [14]. However, neither in [28], [14], or [41] changes of PDGF-B levels have been linked to the RG or YC-1, nor have the effects been associated to any effects of reactive gliosis.

The results of the Examples of this invention indicate that PDGF-B expression was predominantly localized within the cell bodies of the retinal neurons; and specifically expressed by the cells of the retinal GCL and NFL [FIG. 6 (PDGF slides), FIG. 11 (PDGF graph)]. The source of this PDGF-B was next investigated in a series of in vitro experiments in which glial cells were subjected to hypoxic conditions for 72 hours. Such treatment caused a profound increase in PDGF-B at the message and the protein levels. Taken together, these findings show the contribution of Müller cells to retinal PDGF-B and implicate an autocrine mechanism of PDGF-B on Müller cells. The data of the Examples of this disclosure further show that there is intimate association between PDGF-B and RG. The data of the subject-matter presented here of the present invention here prove that in the OIR mouse model ischemia alone is sufficient to induce a marked gliosis in the Müller cell processes of the mouse retina in vivo.

Several studies have critically demonstrated that iNOS is one of the major contributors to NO production in Müller cells; as well as it plays a crucial role in retinal neovascular disease and exhibited that it offers an ideal target for the control of vitreal NV through improvement of the vascularization of the hypoxic retina [62]. Other studies have highlighted the intimate association between GFAP and iNOS [62: unpublished data]. These studies detected the activation of Müller cells in the ischemic area by GFAP immunohistochemistry in the retina of oxygen-induced iNOS+/+ mice. Furthermore, previous investigations have suggested that iNOS expression may serve as an index of RG [6, 50]. Thus, the present results of the disclosure of the invention show that ischemia activates Müller cells, in vitro and in vivo.

The data presented here demonstrate that hypoxia has triggered RG within 72 hours of exposure in vitro [Müller glial cells and retinal neuorosensory cells, FIG. 10], while the ischemic retina has fully developed. RG at P17 in vivo [OIR mouse model FIG. 5E, FIG. 6 (GFAP slides)]. In both models the hallmark of RG, GFAP, is upregulated. This manifestation was associated with the aberrant expression of PDGF-B and iNOS. Increased NO production from its iNOS occurs in different cell types in the eye associated with pathologic conditions such as retinal degeneration, ocular inflammation, cataracts, and diabetes [12]. Moreover, iNOS is known to contribute to superoxide production, while deficiency of iNOS results in less generation of superoxide anion by retina, both in diabetes and in experimental galactosaemia [21]. Several reports have revealed that YC-1 exhibited a potent activity at abrogating the expression of iNOS in macrophages stimulated by lipoteichoic acid in vitro [31]. Prior to the disclosure of the present application, no one has ever used YC-1 on PDGF-B, GFAP, and RG.

The results presented here for the subject-matter of the present invention demonstrate that iNOS plays an important role in the pathogenesis of ischemic retinopathy. Based on these previous observations, the high expression of iNOS in the Müller cells is considered to contribute to the process of retinal degeneration of the ischemic retina after the ischemic insult. However, the mechanism by which the iNOS expressed in the Müller cells and in the retinal ganglion cells causes tissue damage to the ischemic retina remains to be elucidated. iNOS may be the one mediating HIF activation via PI3K/Akt signaling pathway and may be another avenue of intervention [29].

Previous data have demonstrated that one the primary effects of iNOS expression is the inhibition of angiogenesis in the ischemic tissue [62]. The same study has demonstrated that iNOS plays a crucial role in retinal neovascular disease by inducing retinal vaso-obliteration and enhancing pathological intravitreal NV. The same group has noted that iNOS deficiency accelerates revascularization of the avascular retina and significantly reduces vitreal invasion and pre-retinal growth.

According to the results of the Examples of the present invention, administration of YC-1, in the ischemic retinas of P12 and P15 mice have significantly downregulated iNOS expression, when compared with DMSO-injected retinas. Hence, it is established that mice treated with the invention, in particular of YC-1, were rescued from the Ischemia-induced production of iNOS. Moreover, the efficiency of iNOS inhibition by intravitreal administration could also be seen in a significant improvement of intraretinal RV and an inhibition of pathological NV compared with non-treated and DMSO treated ischemic retinas. Taken together, this is good evidence that iNOS activity promotes vessel loss, reduction in the vascular density, and the formation of avascular zones in the ischemic retina.

Hypoxia inducible factor-1 [HIF-1] is a master regulator that controls the transcriptional activation of VEGF signaling and other hypoxia-inducible genes [53]. Like VEGF; PDGF-B is a hypoxia-regulated gene [8]. Previous studies have revealed that YC-1 inhibits HIF-1 and other proangiogenic factors [VEGF, MMP-9, ET-1, and EPO] in the ischemic retinas of the OIR mouse model [16, 18].

Throughout the course of this investigation and the description of the subject-matter of the present invention, it can be shown that targeting hypoxia and HIF-1 signaling maybe considered as a therapeutic modality to target several downstream angiogenic molecules, such as VEGF and PDGF-B, and iNOS which play crucial roles in ischemic retinopathies. Furthermore, the data suggest that via inhibiting HIF-1 signaling and it's downstream angiogenic molecules [VEGF and PDGF-B], the invention, in particular YC-1, may reduce the number or length of filopodia on endothelial tip cells in the OIR mouse model. Moreover, sprouting angiogenesis involves collective migration processes [24]. Tip cells are distinguished by their strong expression of PDGF-B mRNA and VEGFR2 mRNA and protein, implying that tip cells have a distinct gene expression profile [26] and regulate the coordinated processes of neovascular sprouting. These findings would be consistent with the decreased length and number of filopodia in YC-1-treated retinas.

Taken together, the: [i] decrease in the length and number of filopodia in tip cells; and [ii] the attenuation of the message and protein levels of VEGF, and PDGF-B [tip cell marker][26] by treatment with substances and compositions of the invention, in particular YC-1 treatment, collectively show that the invention, in particular YC-1, inhibits growth factors-induced activation of tip cells.

The data presented provide further evidence of tip cell insufficiency by treatment with substances and compositions of the invention, in particular YC-1 treatment. The data shown here clearly demonstrate that the substances and composition of the invention, in particular YC-1, significantly and dose-dependently reduce the number and the total length of filopodia per endothelial tip cell as compared to untreated and DMSO-treated oxygen injured retinas.

The data also reveal that the substances and composition of the invention, in particular YC-1, selectively inhibits NV, while concomitantly promotes physiological RV in a mouse model of OIR. Retinal treatment with the substances and composition of the invention, in particular YC-1, induces the reversal of the vasculature growth to a state that was comparable to the retinas that were grown under normoxic conditions. Taken together, it is demonstrated that the substances and composition of the invention, in particular YC-1, can be exploited as valuable therapeutic modality in the treatment of NV in the ischemic retina. Antagonists of PDGFs may help to reduce scarring, but may also synergize with VEGF antagonists to reduce NV through their antagonism of pericytes, which provide survival signals for endothelial cells of new vessels [2]. Kinase inhibitors that block both VEGF and PDGF receptors are some of the most efficacious drugs for the treatment of ocular NV in animal models [50, 61].

Successful inhibition of GFAP using antisense oligonucleotides has also been reported by other investigators [42, 74]. The findings of the inhibitory effects of the substances and composition of the invention, in particular YC-1, on GFAP synthesis makes the substances and composition of the invention, in particular YC-1, a potential drug in the prevention of RG. This is the first report, which shows the effects of the substances and composition of the invention, in particular YC-1, on RG. In conclusion, administration of the substances and composition of the invention, in particular YC-1, protects neurons in the retina from ischemic injury. The present data indicate that the substances and composition of the invention, in particular YC-1, have potential therapeutic implications by contributing to the rescue process of retinal capillaries in the OIR mouse model.

The substances and the pharmaceutical compositions disclosed herein are suitable for oral, rectal, transmucosal, transdermal, intestinal, parenteral, intramuscular, intrathecal, direct intraventricular, intravenous, intraperitoneal, intranasal, or intraocular, or subcutaneous administration. Also, substances and pharmaceutical compositions can be administered by injections.

For injection, the conjugates presented herein may be formulated in aqueous solutions, preferably in physiologically compatible buffers such as Hank's solution, Ringer's solution, or physiological saline buffer with or without organic solvents such as propylene glycol, polyethylene glycol. For transmucosal administration, penetrants are used in the formulation. Such penetrants are generally known in the art. Aqueous solutions disclosed herein comprise suspensions, solutions or emulsions in oily or aqueous vehicles, and may contain formulatory agents such as suspending, stabilizing and/or dispersing agents.

Pharmaceutical compositions of the present invention may be manufactured by processes well known in the art, e.g., by means of conventional mixing, dissolving, granulating, drageemaking, levigating, emulsifying, encapsulating, entrapping or lyophilizing processes. For oral administration, the substance can be formulated readily by combining the active compounds with pharmaceutically acceptable carriers well known in the art like tablets, lozenges, pills, dragees, capsules, liquids, gels, syrups, slurries, suspensions, solutions or emulsions in oily or aqueous vehicles. Pharmacological preparations for oral use can be made using a solid excipient to obtain tablets or dragee cores. Suitable excipients are lactose, sucrose, mannitol, sorbitol, maize starch, wheat starch, rice starch, potato starch, gelatin, gum tragacanth, methyl cellulose, hydroxypropylmethyl-cellulose, sodium carbomethylcellulose, and/or physiologically acceptable polymers such as polyvinylpyrrolidone (PVP).

For administration by inhalation, the substances and pharmaceutical compositions can be administered as an aerosol spray.

### BRIEF DESCRIPTION OF THE DRAWINGS

FIG. 1 Analyses of Retinal Vascular Development in the Normal Retinal Vasculature and the Neovascular Retina. Mice were perfused with fluorescein-labeled dextran on P2 [A], P4 [B], and P7 [C]. P stands for ‘postnatal birth’ and therefore, P2 indicates a retinal flatmount that was extracted from a 2-day old mouse, P4 from a 4-day old mouse, and so forth. In another group of mice, retinal NV was induced in newborn mice as described in methods, and mice were perfused with fluorescein-labeled dextran on P12 [D], P15 [E], and P17 [F]. Retinal flat mounts were examined by fluorescence microscopy. At P2 only budding superficial vessels were observed occupying a single plane around the optic disc. At P4, the margin of the developing blood vessels on the surface of the retina is located halfway between the optic nerve and the peripheral edge of the retina. At P7, superficial vessels cover about 80% of the retina.

FIG. 2

A. Suppression of vaso-obliteration and retinal NV with YC-1 treatment. Images of the non-treated OIR retinas and the DMSO-treated retinas at P17 both have exhibited partial RV from the periphery inward after return to normoxic conditions at P12, accompanied by the formation of pathologic new vascular tufts. Please note the presence of the capillary-free zones in the central segment of the retina, and the presence of severe vessel tortuosity, while normoxic retinas showed no presence for vaso-obliterations. YC-1 treatment led to the suppression of vaso-obliteration, whereas vaso-obliteration was unimpeded with the DMSO-treatment. The vascular integrity and the density of vessels within the retinal tissues of the normoxic and YC-1-treated retinas shared common similarities and looked relatively normal and lacked the tortuosities. Whereas ischemic and DMSO-treated retinas have both exhibited disrupted vascular integrity and a significant degrees of vasculopathy. [n=15 per group].

B. Quantification avascular area. Images exhibit the percentage of the capillary-free area/retina of mice with different treatments. Oxygen treatment resulted in a significant increase of the avascular area as compared with mice that were kept under ambient conditions [**P<0.01]. Treatment with YC-1 promoted a significant de novo vessel growth in the avascular retina [**P_{<}0.01] as compared to DMSO-treated controls. [n=15 per group].

C. Quantification of pre-retinal neovascular tufts. Oxygen treatment resulted in a significant increase of BV tufts number and area of tufts as compared to mice that were kept under ambient conditions [**P<0.01]. Treatment with YC-1 significantly reduced the number of BV tufts and the area of tufts [**P<0.01] as compared to DMSO-treated controls. [n=15 per group]. Scale bar: 60

D. Quantification of blood vessel tortuosity. Tortuosity was significantly increased in nontreated ischemic retinas [**P<0.01], and DMSO-treated retinas [**P<0.01] as compared to normoxic retinas. Tortuosity was significantly decreased in YC-1-treated retinas as compared with DMSO-treated retinas [**P<0.01]. Graph represents arteriole tortuosity indices [TI] of retinas from all groups. [n=15 per group]. Scale bar: 60 μm.

E. Quantification of retinal vasodilation. Each retina was digitized with at X20 objective and tiled to present the entire preparation. Graph represents the quantification of averaged diameter of BVs from retina flat mounts from mice of different groups. Values are mean±SEM of 6 independent samples. **P<0.01 vs. DMSO-treated group. [n=15 per group]. Scale bar: 60 μm.

F. Quantification of preretinal NV. Mice kept in room air or exposed to a cycle of hyperoxia and room air (ROP). H&E-stained section from P17 wild-type, animals exposed to OIR, DMSO-injected animals, and YC-1-treated animals are shown. The number of vascular cell nuclei present on the vitreous side of the retina penetrating the INL was determined in a masked fashion. Intravitreal injection of YC-1 significantly reduced (**P<0.01) the number of pre-retinal nuclei as compared with the DMSO-treated ischemic retinas. The graph below shows the mean number of vascular cell nuclei, projecting into the vitreous of eyes from all mouse groups. Data in each bar are the mean number of vascular cell nuclei in five eyes of four mice. Values are presented as mean±S.E.M. Statistical significance was analyzed usingANOVA. [n=15 per group]. Scale bar: 110 μm.

FIG. 3

A. Quantification of vascular area. Images exhibit the percentage of vascular area/retina of mice with different treatments. Oxygen treatment resulted in a decrease of the vascular area as compared to untreated mice [**P<0.01]. YC-1 treatment promoted a significant de novo vessel growth in the avascular retina [**P<0.01] as compared to DMSO-treated group. [n=15 per group]. The retinal vascular morphology and densities of vessels within the non-treated normoxic retinas were similar to retinal vasculature of YC-1-treated retinas. While animals that were treated with DMSO exhibit dramatic vasculopathies, which resembled the ocular pathologies that were demonstrated by the ischemic retinas. [n=15 per group].

B. Quantitative assessment of retinal vascular density at P17. A CD31-stained tissue sections prepared from all mouse groups are shown. For quantitative assessments, staining intensities were quantified as described in the Materials and Methods. The vascular density is significantly (**P<0.01) greater in the YC-1-treated retinas compared with DMSO-treated retinas. Please note the increased number of vessels in the superficial and deep layers of normoxic and YC-1-treated retinas. The graph below, which shows the number of endothelial cells per reticle square in all mice groups, has demonstrated that there was a significant increase (**P<0.01) in the vascular density of YC-1-treated retinas, as compared to the DMSO-treated group. [n=15 per group].

FIG. 4 the Influence of YC-1 on filopodia number and length. Images exhibit a higher magnification of growing tips of BVs that reveal the tip of a sprout with multiple filopodia growing at multiple angles including both along and above the plane of the section. Image of tip cells along the leading edge of vascularization from; [A] Non-treated ischemic retinas; [B] DMSO-treated retinas; [C, D, E, and F] represent retinas from animals that were injected with YC-1 [25-100 μl]. Filopodia lengths and number/tip cell were not influenced by DMSO treatment. Whereas treatment with YC-1 significantly and dose-dependently inhibited the number and the length of filopodia/endothelial cell. [n=5 per group].

FIG. 5 Measurements of the average change in filopodia length and number.

A. Filopodia from DMSO-treated retinas had approximately similar lengths as the nontreated ischemic retinas. YC-1 treated animals exhibited a significant and a dose dependent decrease in filopodia lengths as compared to DMSO-treated retinas. [ANOVA, ***P<0.001; **P<0.01 between YC-1 and DMSO]. There were 24 tip cells/retina that were averaged. [n=5 per group].

B. DMSO has no influence on the number of filopodia/tip cell. Whereas YC-1 exhibited a significant and a dose-dependent decrease in filopodia number/tip cell. [ANOVA, ***P<0.001; **P<0.01 between YC-1 and DMSO]. There were 24 tip cells/retina that were averaged. [n=5 per group].

C. Sequence for the primer sets used for the quantitative Real-Time PCR analysis.

D. through. H. Real time RT-PCR analysis, in vivo. The mRNA levels of VEGF [FIG. 5E], GFAP [FIG. 5F], PDGF-B [FIG. 5.G], and iNOS [FIG. 5F] were increased in the non treated ischemic retinas, while non-treated normoxic retinas exhibited extremely low levels. Treatment of ischemic retinas with dual injections of YC-1 resulted in a significant knockdown of VEGF [***P<0.001], PDGF-B [**P<0.01], GFAP [**P<0.01], and iNOS [***P<0.001] gene expression when compared with DMSO treated controls. Ischemic, DMSO or YC-1 treatment didn't have any influence on HIF-1α gene expression levels. [ANOVA; Mean±SEM of mRNA level normalized to β-actin were calculated, [***P<0.001 and **P<0.01, as compared to DMSO-treated retinas]. Data are representative of 3 independent experiments.

FIG. 6 Immunohistochemical analysis, in vivo. Photomicrographs of retinas from various OIR groups that were immunostained for HIF-1, VEGF, GFAP, PDGF-B, and iNOS. The expression of all proteins was upregulated in the non-treated ischemic and DMSO-treated groups, compared with non-treated normoxic group. While all protein immunoreactivities were downregulated in the YC-1-treated group, compared with DMSO-treated groups. Data are representative of 3 independent experiments. Scale bar: 140 μm.

FIG. 7

A. Morphological analysis of rMC-1 and R28 cells. Hypoxia altered the morphology of R28 and rMC-1 cells. Under normoxia, R28 cells adopt a neuronal morphology, with neurite-like projections, and their rate of division was reduced. While rMC-1 cells are large and flat cells. Hypoxic exposure for 72 hours induced apparent hypertrophic morphological changes in both cell types. Data are representative of 3 independent experiments. Scale bar: 50 μm.

B. Coculture models. [i] Cell proliferation assay model. [ii] Cell migration assay model.

FIG. 8

A. And B. Effects of YC-1 on the proliferation and migration of ECs in a coculture system. hRMVECs growth curves from four groups were depicted. Coculture group had a higher proliferation and migration rate of hRMVECs cells than that of hRMVECs solo[**P<0.01]. Hypoxia significantly increased hRMVECs proliferation and migration rate in the coculture system [**P<0.01, rMC-1/hypoxia vs. rMC-1/normoxia]. After rMC-1 cells were treated with YC-1 [100 μM], the proliferation and the migration rate of hRMVECs were significantly inhibited compared with the rMC-1/hypoxia group. [**P<0.01, YC-1-treated rMC-1/hypoxia vs. nontreated rMC-1/hypoxia]. Data are representative of 3 independent experiments.

C. Western blot analysis. Protein expression levels were elevated markedly in the nontreated hypoxic cells. In YC-1-treated hypoxic cells, HIF-1α; VEGF; GFAP; PDGF-B; and iNOS protein expression were significantly decreased in a dose-dependent fashion, compared with DMSO-treated hypoxic cells. Statistical significance was determined by ANOVA [**P<0.01]. Data are representative of 3 independent experiments.

FIG. 9 Immunofluorescence analysis of GFAP expression, in vitro. rMC-1 and R28 cells were immunostained with anti-GFAP antibody. Intense staining was considered a positive signal, which indicate GFAP immunostaining. Under hypoxia, non-treated cells exhibited extremely high GFAP immunoreactivity. Treatment of cells with YC-1 [25-100 μM] under hypoxia for 48 hours resulted in a dose-dependent inhibition of GFAP expression. Images are representatives of 3 independent experiments. Scale bar: 40 μm.

FIG. 10 Real Time RT-PCR analysis, in vitro. R28 and rMC-1 cells were cultured under normoxic and hypoxic conditions. The mRNA levels of VEGF, GFAP, PDGF-B, and iNOS were upregulated in all non-treated hypoxic cells; while normoxic cells exhibited remarkable low mRNA levels. Treatment of hypoxic R28 and rMC-1 cells with various concentrations of YC-1 resulted in a significant inhibition of VEGF, GFAP, PDGF-B, and iNOS mRNA expression as compared to the DMSO-treated controls. The message level of HIF-1α was not influenced by hypoxia, DMSO, or YC-1 treatments as compared to normoxic cells. ANOVA was used for statistical analyses. Mean±SEM of mRNA level normalized to β-actin were calculated, [***P<0.001 and **P<0.01 as compared to DMSO-treated hypoxic control. Data are representative of 3 independent experiments].

FIG. 11

A. Viability assay. Viable rMC-1 and R28 cells in culture comprised the majority of cells that were detected by a DAPI exclusion test after incubation with YC-1. A significant nonviability staining was detected when cells were treated with Doxorubicin. Data are representative of 3 independent experiments.

B. Assessments of retinal immunohistochemical analysis. Values obtained from at least 5 retinal fields were used to calculate the average pixel intensity value/retina. Bar graphs exhibit the area of staining of HIF-1, VEGF, GFAP, PDGF-B, and iNOS in all four groups [Shown in FIG. 6]. Values [mean±SEM], from 3 separate experiments from at least 10 images from 4 different eyes/group. [***P<0.001 and **P<0.01 as compared to DMSO-treated retinas]. Data are representative of 3 independent experiments.

C. Assessments of the cellular immunocytochemical analysis of GFAP. Graphs showing the intensity of GFAP staining in rMC-1 and R28 cells after treatment with YC-1 relative to that measured in DMSO-treated hypoxic control [shown in FIG. 10]. The areas of staining to GFAP/μm2 in all four groups were measured. Values, shown as the mean±SEM, from 3 separate experiments.

FIG. 12. Densitometric analysis of Western Blot. Graphs indicate the densitometric analysis. Relative ratio represented the intensities of HIF-1α, VGEF, GFAP, PDGF-B, and iNOS protein expressions in rMC-1 and R28 cells [shown in FIG. 8C] relative to those of β-actin expression, whereas the relative ratio of hypoxia control was defined as 100. Values, shown as the mean±SEM, from 3 separate experiments with a total sample size of 6. [**P<0.01 as compared to DMSO-treated hypoxic control].

### EXAMPLES

### Example 1

The following general methods were used in the subsequent Examples.

### 1. Reagents

YC-1 was purchased from A.G. Scientific [San Diego, Calif.] and dissolved in sterile dimethyl sulfoxide [DMSO]. Fluorescein isothiocyanate [FITC]-dextran 2,000,000 was purchased from Sigma-Aldrich [St. Louis, Mo.]. Monoclonal mouse anti-HIF-1α [clone H1α67] and monoclonal rabbit anti-VEGF antibodies were both purchased from Millipore [Billerica, Mass.]. Rabbit Anti-PDGF-B polyclonal antibody was obtained from Abbiotec [San Diego, Calif.]. The GFAP labeling was carried out by a polyclonal antibody [Sigma, catalog number G9269] during immunohistochemistry or a monoclonal GFAP antibody produced in mouse [Sigma, catalog number G3893] for Western Blot analysis. For iNOS Western blot and Immunofluorescence staining; monoclonal mouse anti-iNOS antibody was purchased from Abcam [Cambridge, Mass.]; whereas, monoclonal mouse anti-iNOS antibody, which was purchased from BD Biosciences [San Diego, Calif.], was used in all immunohistochemistry staining. Polyclonal rabbit anti-β-actin antibody was purchased from MBL Intl [Woburn, Mass.].

### 2. Tissue Culture

A transformed Müller cell line [rMC-1] were grown in cell cultures in DMEM supplemented with 15% FBS, as well as with a fungicide mixture and 0.5% gentamicin in a humidified atmosphere of 5% CO2/95% air. Medium was changed every 2-3 days, and cells were grown to confluence in a 150-mm dish. Cells were split into 60-mm dishes and were used in the experiments when confluent. R28 cells are immortalized rat retinal neurosensory/neuoroglial progenitor cells, by transfection with Adenovirus 12S E1A into the neonatal retinal tissue. R28 cells express genes characteristic of neurons, as well as functional neuronal properties. R28 cells were cultured in DMEM/F12 medium in a 1:1 mixture, supplemented with 5% FBS, 1.5 mM L-glutamine, 7.5 mM sodium pyruvate, 0.1 mM nonessential amino acids, 1×MEM, 0.37% sodium bicarbonate and 10 μg/ml gentamicin. Cells were incubated at 37° C. in the presence of 5% CO2. Human retinal microvascular endothelial cells [hRMVEC][40,000 cells/well] were seeded in a 96-well plate and allowed to adhere overnight and incubated at 37° C. in normoxia. Cells were then cultured in 150 μl of CS—C medium supplemented with 10% FBS and in the presence of 1-100 μM YC-1 or DMSO [0.2%].

### 3. In Vitro Hypoxia

Cells were placed in airtight chambers [BioSpherix, Redfield, N.Y.] and the O2 tension was maintained at 1.2% by using Pro-Ox Model 110 O2 regulator [BioSpherix, Redfield, N.Y.]. The chamber was purged with a gas mixture of 5.32% CO2, and 93.48% N2. We have confirmed that these conditions do not affect viability of the cell cultures [data not shown].

### 4. Morphological Analysis of Cells

rMC-1 and R28 cells were cultured in their appropriate media containing YC-1 [25-100 μM] or DMSO [0.2%]. The cells were incubated under hypoxic or normoxic conditions for 72 hours at 37° C. Cells morphology was examined under Zeiss Axiovert 135 [Thornwood, N.Y.] and examined triplicates at [X100] using inverted bright field microscopy.

### 5. Cell Proliferation in a Co-culture System Model

To test the influence of Móller cells [rMC-1] on the hRMVECs proliferation, rMC-1 [2×105 cells cm_{—}2] were plated in a transwell insert [Millipore, Billerica, Mass.] with 0.4 mm pores and allowed to adhere overnight in 150 μL of CS—C complete medium [Cell Systems, Kirkland, Wash.], supplemented with 10% FBS and incubated at 37° C. under normoxic [5% CO2/95% air]. The rMC-1 cells were then incubated under normoxic [5% CO2/95% air], or hypoxic conditions. To establish hypoxic conditions, cells were placed in airtight chambers [BioSpherix, Redfield, N.Y.] that were flushed with a gas mixture of 5% CO2 and 95% N2. Oxygen concentrations within these chambers were maintained at 1.2% using Pro-Ox Model 110 O2 regulators. After treating the r-MC-1 cells with YC-1 [100 μM] for 24 hours, the insert was then placed into 24 well-plates, in which hRMVECs were plated at 5×103 cells cm⁻² and allowed to adhere overnight [FIG. 7B]. hRMVECs proliferation was evaluated using 3, [4,4-dimethylthiazol-2-yl]-2,5-diphenyl tetrazolium bromide [MTT] colorimetric assay [Mosmann,1983], at 24, 48, 72, and 96 hours after coculture. During the last 4 hours of each day, 100 ml of 5 mg/ml MTT [Millipore, Billerica, Mass.] was added in each well. Formed Formazan crystals were dissolved in 600 ml DMSO and optical density was recorded at 492 nm. Experiments were performed on at least three independent occasions. Data were presented as a percentage of negative control proliferation with p<0.01 being significant.

### 6. Cell Migration in a Co-culture System Model

Migration assay of hRMVECs was carried out using the transwell insert with 8 mm pore size. The inserts were coated with Extracellular matrix [ECM] [Millipore, Billerica, Mass.] and airdry up. Chemotaxis was induced by the control r-MClcells or the r-MC1 cells that were treated with YC-1 [100 μM], which were situated in the lower compartment. hRMVECs suspension [final concentration, 5×104 cells/well] was added to the upper compartment [FIG. 7B]. After incubated for 24 hours, the filters were washed and then fixed and stained with crystal violet [0.5% crystal violet and 20% methanol] for 30 min at room temperature. The filters were washed with distilled water, and the cells on the upper surface of the inserts were wiped with a cotton swab. The number of cells per field that migrated to the lower surface of the filters was determined microscopically. Five randomly chosen fields were counted per filter. Data were presented as a number of migrated cells [**P<0.01].

### 7. Quantitative RT-PCR by Molecular Beacon Assays

mRNA levels for HIF-1, VEGF, PDGF-B, GFAP and iNOS were quantified by Real time RTPCR. We used the primers summarized in [FIG. 6C] for RT-PCR. Gene-specific molecular beacons and primers were designed to encompass the genes of interest, with beacon's annealing site to overlap with the exon-exon junctions for additional specificity [Beacon Designer 6.0, Premier Biosoft International, Palo Alto, Calif., USA]. Threshold cycle [Ct] values for the different samples were utilized for the calculation of gene expression fold change using the formula 2 to the minus power of delta delta ct. Fold changes in the HIF-1, VEGF, PDGF-B, GFAP and iNOS gene relative to the β-actin endogenous control gene were determined by the following equation: fold change=2−Δ[ΔC T], where change in threshold cycle [ΔCT]=CT [gene of interest]−CT [β-actin] and Δ [ΔCT]=ΔCT [treated]−ΔCT [untreated].

### 8. Western Blot

Müller cells [rMC-1] and R28 cells were seeded overnight in 6-well plates [105 cells/well]. Cells were treated with either YC-1 [25-100 μM] or DMSO [0.2% v/v] for 48 hours under normoxic or hypoxic environments. Reactions were terminated by addition of lysis buffer [Cell Signaling, Beverly, Mass.]. Protein content of the cell lysates was determined according to the Bradford method [Bio-Rad, Hercules, Calif.]. Aliquots [40 μg] of whole-cell lysates were separated on 7.5% SDS-PAGE, and electro-transferred onto polyvinylidene membranes [Amersham Pharmacia Biotech, Little Chalfont]. After blocking with 5% nonfat dry milk in TBS-T, the blots were incubated overnight with anti-iNOS [at 1:100] and anti-β-actin [internal control] antibodies. Then blots were washed 3×10 min washes in PBS/tween and subsequently incubated with peroxidase-conjugated anti-mouse IgG secondary antibody at 1:3000. The signals were obtained by enhanced chemiluminescence [Amersham Biosciences], and visualized by exposure to X-ray film. Upon completion of chemiluminescence, equal lane loading was checked by Ponceau S Solution [Sigma, St. Louis, Mo.]. X-ray films were scanned with a computer-assisted densitometer [model G-710; Bio-Rad] to quantify band optical density [Quantity One software; Bio-Rad].

### 9. GFAP Immunofluorescence

Müller cells [rMC-1] and R28 cells [2×104 cells per well] were grown on 8-well chamber slides and cultured in 300 μl of their growing media, which contained YC-1 [25-100 μM] or DMSO [0.2% v/v] and incubated under normoxia or hypoxia for 48 hours at 37° C. YC-1 or DMSO was added 5 minutes prior to the hypoxic incubation. The cells were fixed 48 hours later with 3.7% paraformaldehyde and permeabilized with 0.2% Triton™ X-100 in PBS. The cells were incubated for two hours with anti-GFAP antibody. Negative control experiments consisted of omission of the primary antibody. Cells were then incubated with HRP-conjugate working solution, followed by the addition of the Tyramid solution [TSA Kit#2 and #4 for rMC-1 and R28 staining, respectively] at 1:100 dilutions [Molecular Probes, Carlsbad, Calif.]. Digitized images were acquired utilizing AxioVision software [Zeiss Axiovert 135 and AxioCam]. Intensity values of immunofluorescence staining of GFAP in rMC-1 and R28 cells was analyzed and quantified using Metamorph™ imaging analysis software version 6.0 [Universal Imaging, Sunnyvale, Calif.]. The staining intensity in our series ranged from a weak blush to moderate or strong. The amount of cells staining with the antibody was further categorized as focal [<10%], patchy [10%-50%], and diffuse [>50%]. For semiquantitative analysis, focal and/or weak staining was considered equivocal staining, and patchy or diffuse staining was subcategorized as either moderate or strong.

### 10. Animals and Experimental Design

C57BL/6J mice, from Jackson Laboratory [Bar harbor, ME] were used in these experiments. All animal protocols were approved by the Institutional Review Board and conformed to the ARVO Statement for the Use of Animals in Ophthalmic and Vision Research statement of the Association for Research in Vision and Ophthalmology. Mice were divided into four separate groups; [1] Non-treated mice grown under ambient conditions [negative control]; [2] nontreated hyperoxia-exposed mice [positive control]; [3] DMSO-treated hyperoxia-exposed mice [sham-treated]; and [4] YC-1-treated hyperoxia-exposed mice [drug-treated].

### 11. Mouse Model of Oxygen Induced Retinopathy

Retinal NV was induced in newborn mice as described previously [64]. Briefly, P7 mice were exposed with their nursing mother, for 5 days [between P7 and P12] to hyperoxic conditions, by incubating them in an airtight chamber [PROOX 110 chamber O2 controller; Biospherix Ltd., Redfield, N.Y.] ventilated by a mixture of O2 and air to a final oxygen fraction of 75±2%. These incubation conditions induced vaso-obliteration and subsequent cessation of vascular development in the capillary beds of the central retina [64]. At P12, the mice were allowed to recover in normal room air conditions and maintained for another 5 days [till P17], the day in which peak disease occurs. A condition of relative hypoxia resulted between P12 and P17, and extensive retinal NV developed in 100% of the mice. Age-matched animals with the hyperoxia-exposed groups were maintained identically, except they were exposed to room air [2]% O2, 79% N2] for the entire duration of the experiment. All animals were examined and sacrificed on the same days.

### 12. Intravitreal Drug Injections

A group of hyperoxia-exposed animals [n=15] were injected intravitreally [into both eyes] at P12 and P15 with 3 μl of YC-1 [100 μM] [drug-treated group]. Another group of hyperoxiaexposed mice [n=15] were injected intravitreally [into both eyes] at P12 and P15 with 3 μl of DMSO [0.2% [v/v]] [mock-treated group]. Non-treated mice grown under ambient conditions, non-treated hyperoxia-exposed mice, DMSO-treated hyperoxia-exposed mice and YC-1-treated hyperoxia-exposed mice, were all examined at different critical time points for qualitative assessment of the retinal vasculature by fluorescein angiography.

### 13. Retinal Fluorescein Angiography and Visualization of Retinal Vascularization

Deep anesthesia was induced by intraperitoneal injection of ketamine [1%], xylazine [0.1%], and sodium chloride [0.9%] in a concentration of 0.1 mL/10 g mouse body weight. Sternotomy was performed, the mice were perfused through the left ventricle with 600 μL of high-molecular-mass [2×106 Da] FITC-dextran in PBS [50 mg/ml], which was allowed to circulate for 2 minutes before the animals were euthanatized and the eyes enucleated and the flat mounts were prepared. Subsequent to retinal extraction, all retinas were fixed in 4% paraformaldehye for 24 hrs at 4° C. A dissecting microscope was used to dissect the cornea with a circumferential limbal incision, followed by removal of the lens and vitreous. Microscissors were used to make four radial incisions of the retinal eyecup in order to prepare retinal flat mounts on glass slides. Flat mounts were immersed in Aquamount mounting medium [Polysciences, Warrington, Pa.], coverslips were carefully placed over the retina, and the edges of the coverslips were sealed.

### 14. Quantification of Retinal Vascularization, Avascular Zone, Neovascular Tufts

Images of the perfused retinas were captured with a fluorescence microscope [Zeiss Axiovert 135, Thornwood, N.Y.], and images were captured in a digital format in a 10× and 100× objectives using a digital camera [AxioCam, NY]. The extent of retinal NV was then quantified by Metamorph™ imaging software [Universal Imaging, Sunnyvale, Calif.]. Briefly, the entire retina was outlined to distinguish the total retinal area of each eye. Then, the images were thresholded to emphasize only the FITC-perfused vessels. This permitted the measurement of total BV area of each retina and the percentage of each retina that is engrossed with BVs. The entire mounted retinas were analyzed in a masked fashion [three observers] to minimize sampling bias. The total vascularized area was then normalized to the total retinal area and the percentage of the retina covered by vessels was calculated. The vascular area was quantified by setting a threshold level of fluorescence, above which only vessels were captured [density slicing]. The total surface area of the retina was outlined using the outermost vessel of the arcade near the ora serrata as the border. The central capillary dropout area was quantified from the digital images in masked fashion. For quantitative measurements, z-series (every 10 μm) were captured and compiled, and pixel intensities of identical quadrants from each retina were compared and the percentages were calculated.

### 15. Determination of Retinal Vessels Tortuosities

The index of tortuosity [TI] for arteries and veins was defined as the path length of the vessel divided by the linear distance from the vessel origin to the reference circle [31]. Vessels were also marked from their branching point to the reference circle, and the total number of branching points [arteries and veins], i.e. the number of retinal vessels within this area was automatically calculated. Arteries were distinguished from veins by their smaller caliber and brighter appearance.

### 16. Quantitative Measurements of Vessel Diameters

The diameters of major retinal vessels were measured at two disc diameters from the center of the optic disc in monochromatic images recorded before AO injection. Each vessel diameter was calculated in pixels as the distance between the half-height points determined separately on each side of the density profile of the vessel image and converted into real values using the calibration factor. The averages of the individual arterial and venous diameters were used as the arterial and venous diameters for each mouse.

### 17. Quantification of Preretinal NV

Quantification of preretinal NV on P17 mice was performed as previously described [67]. Briefly, 6-μm-thick serial sections were stained with hematoxylin and Eosin (H&E), then examined and scored in masked fashion for the presence of all nuclei extending beyond the ILM into the vitreous from the retina, by using light microscopy (Zeiss Axiovert 135, Thornwood, N.Y.). Efficacy of treatment was calculated as the percentage of the average number of nuclei/section in the eyes of YC-1-treated mice vs. DMSO-treated retinas. A minimum of 10 sections at least 40 μm apart were evaluated and counted per eye and then averaged. The average neovascular nuclei ±SEM per section per eye was used in the statistical analysis. The neovascular score was defined as the mean number of neovascular nuclei/section found in 10 sections/eye.

### 18. Morphometric Assessment of Vascular Density in the Immunohistochemically Stained Retinal Tissue Sections

Retinas were harvested at various time points and fixed with 4% paraformaldehyde (PFA) and methanol followed by blocking in 50% FBS/20% normal goat serum for 1 hour at room temperature. To stain retinal vasculature, retinas were incubated with anti-CD31 antibody. The sections were washed with TBST and incubated with EnVision Polymer HRP secondary antibody (DAKO, Carpinteria, Calif.) for 30 minutes. Light microscopy (Zeiss Axiovert 135, Thornwood, N.Y.) was employed and images were acquired using a digital camera (AxioCam, N.Y.) at X63 objectives. Quantitation of PECAM/CD-31 immunostaining was processed morphometrically using Metamorph™ imaging software [Universal Imaging, Sunnyvale, Calif.] to determine the area of positive staining for YC-1-treatedt vs. control group. The values were processed for statistical analyses (Mann-Whitney U test). All results were expressed as meant S.E.M. Differences were considered statistically significant at **P<0.01.

### 19. Immunohistochemistry

Mouse retinas were dissected and prepared for immunohistochemical analysis, fixed in 4% paraformaldehyde in 0.1 M PBS for 15 min at room temperature and embedded in paraffin, sectioned [5 μm]. Tissue sections were deparaffinized, hydrated, and later exposed to heat induced antigen retrieval using a microwave oven [three 5-minute cycles in citrate buffer, pH 6.0], endogenous peroxidase was abolished with methanol, and hydrogen peroxide and nonspecific background staining was blocked by incubating the tissue sections for 5 minutes in normal swine serum. Subsequently, all slides were washed three times in PBS, and incubated for 1 hour with primary anti-[HIF-1α, VEGF, PDGF-B, GFAP, iNOS, and β-actin] antibodies. The sections were washed with TBST and incubated with EnVision Polymer HRP secondary antibody [DAKO, Carpinteria, Calif.] for 30 minutes. All slides were stained with DAB solution and counterstained with hematoxylin. Slides were cover slipped [Permount; Fisher Scientific, Fairlawn, N.J.] and examined by light microscopy. Negative controls were performed by omitting the primary antibodies. Sections were photographed under a microscope [Zeiss Axiovert 135, Thornwood, N.Y.], and images were acquired a digital camera [AxioCam, N.Y.]. All retinas were examined at X60 objective. The staining intensity in our series ranged from a weak blush to moderate or strong. The amount of cells staining with the antibody was further categorized as focal [<10%], patchy [10%-50%], and diffuse [>50%]. For meaningful semiquantitative analysis, focal and/or weak staining was considered equivocal staining, and patchy or diffuse staining was either subcategorized as either moderate or strong staining. All Immunohistochemical analyses were measured by Metamorph digital image software [Molecular Devices, Sunnyvale, Calif.].

### 20. Statistical Analysis

Analysis was performed utilizing ANOVA for multiple variables and with t tests for comparison of 2 groups with normal distribution. For the determination of BV tortuosities, statistical significance was defined as *P<0.01; **P<0.005, while P value was obtained by utilizing Bonferroni t-test analysis. For the analysis of the Real Time RT-PCR data; immunohistochemistry data; Western Blot data, analysis was performed with ANOVA for multiple variables and with t-tests. Data are expressed as mean±SEM from at least 3 independent experiments. Significance was defined as *P<0.05; **P<0.01; ***P<0.001.

### Example 2

### Analyses of the Retinal Vasculature and the Progression of Retinal NV

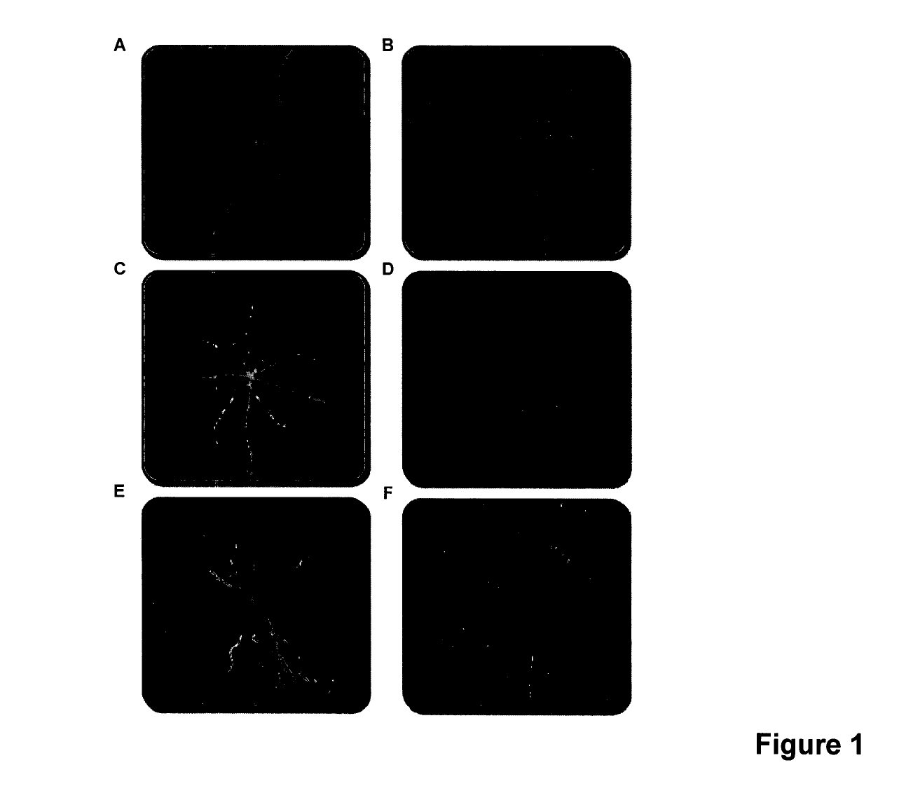

Mice were perfused with fluorescein-labeled dextran on P2 [A], P4 [B], and P7 [C]. P stands for: postnatal birth and therefore, P2 indicates a retinal flatmount that was extracted from a 2-day old mouse, P4 from a 4-day old mouse, and so forth. In another group of mice; retinal NV was induced in newborn mice as described in methods, and mice were perfused with fluorescein-labeled dextran on P12 [D], P15 [E], and P17 [F]. Retinal flat mounts were examined by fluorescence microscopy. At P2 only budding superficial vessels were observed occupying a single plane around the optic disc [FIG. 1; P2]. In P4 wild type mice, the retinal vasculature was organized uniformly and was evenly spaced over the superficial retina and vessels grew into the deeper layers of the retina [FIG. 1; P4]. The vascular density and the number of branch-surrounded spaces increased at P4. In addition, the margin of the developing BVs on the surface of the retina is located halfway between the optic nerve and the peripheral edge of the retina. This is followed by development of a capillary network at P7. During P7, superficial vessels cover about 80% of the retina [FIG. 1; P7]. At this stage sprouts from superficial vessels begin to grow into the retina to form the intermediate and deep capillary beds. In addition, the hyaloidal vessels start to regress. Over the course of the next week, the primary superficial network extended toward the periphery, reaching the far periphery at P12. Angiogram of P12 FITC-dextran-perfused retinal flat mounts of the OIR model preparations displays the effects of 5 days of hyperoxic-exposure [FIG. 1; P12]. On return to normoxia at P12, a relative state of ischemia in the poorly vascularized retina is associated with the excessive re-growth of superficial vessels, leading to abnormal sprouting at the interface between retina and vitreous. Retinas at P12 exhibit typical signs of central non-perfusion of the retina and a drastic regression in the vascular network, leaving only the major vessels and practically no capillary network. The peripheral retina still showed evidence of a vascular network, but, in general, the deep vascular plexuses had completely failed to form. By P15 the retinal ischemia initiates an aggressive neovascular response at the interface of the perfused retinal periphery and the ischemic central capillary beds [FIG. 1; P15]. On P17, the vascular network of the O2-injured retinas was significantly altered as demonstrated by an increase in retinal NV. These retinas displayed the features indicative for a strong ongoing vasoproliferative response: central capillary-free regions, vessel tortuosity and blood vessel tufts [FIG. 1; P17].

### Example 3

### YC-1 Suppresses Retinal NV

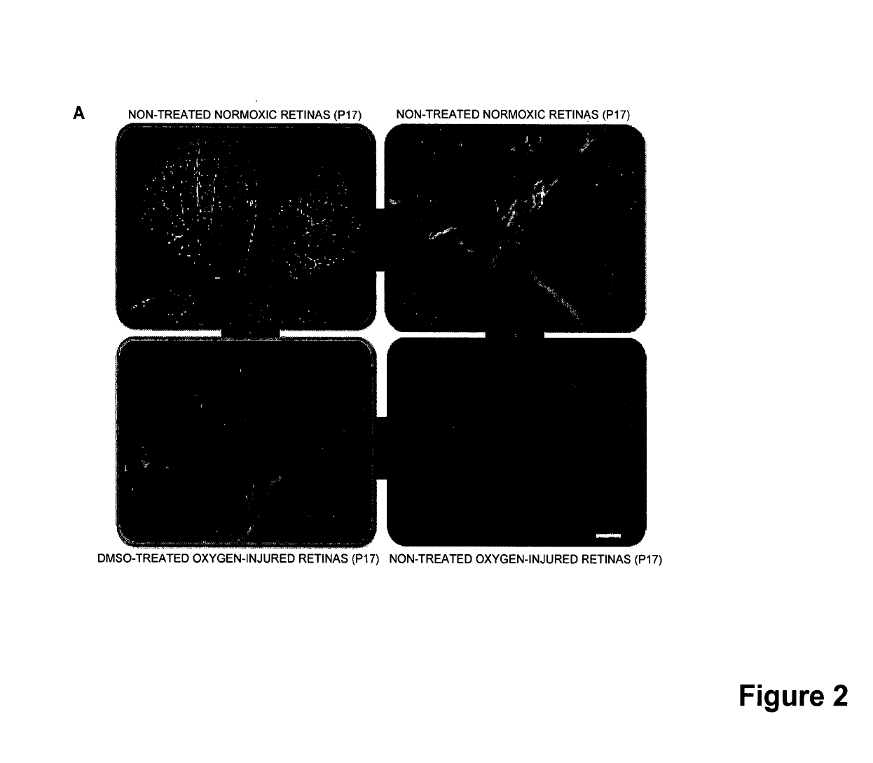

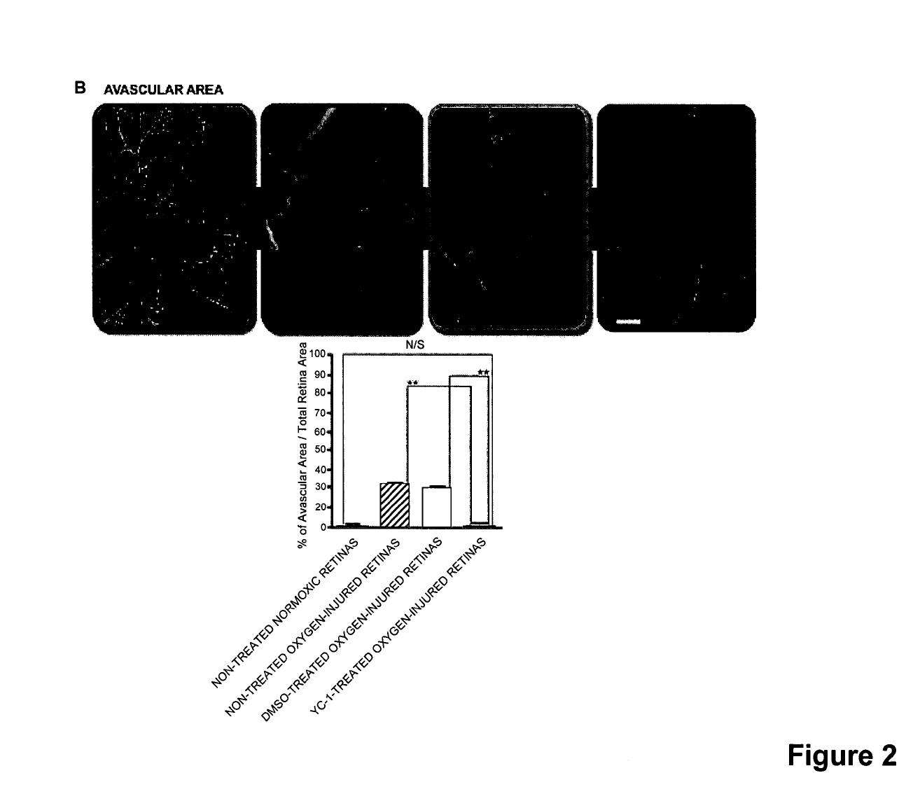

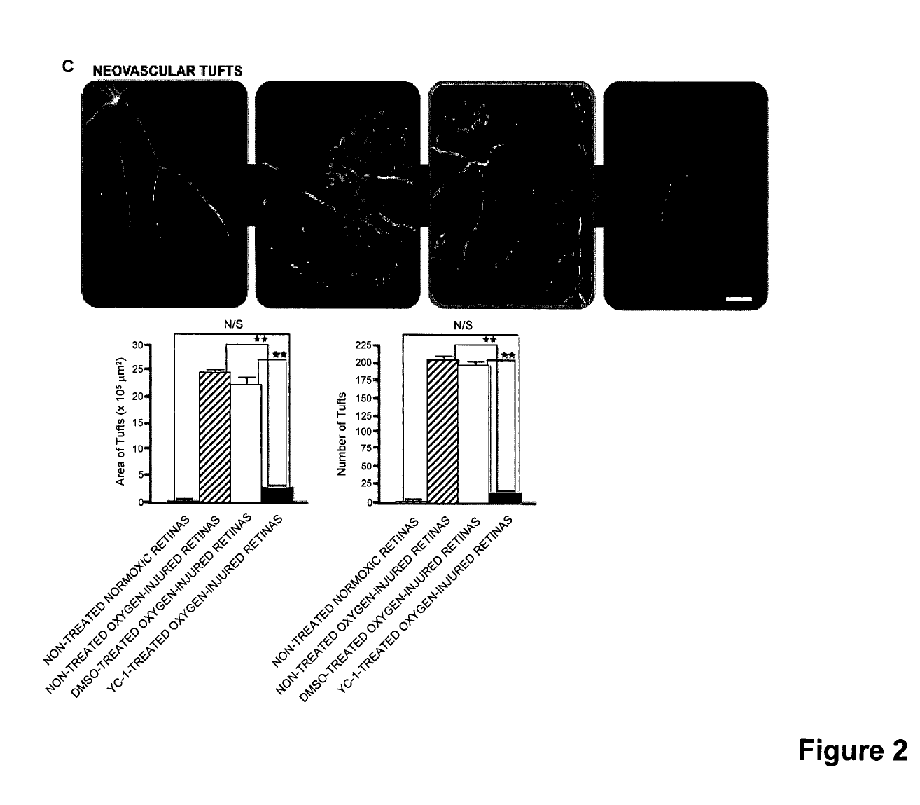

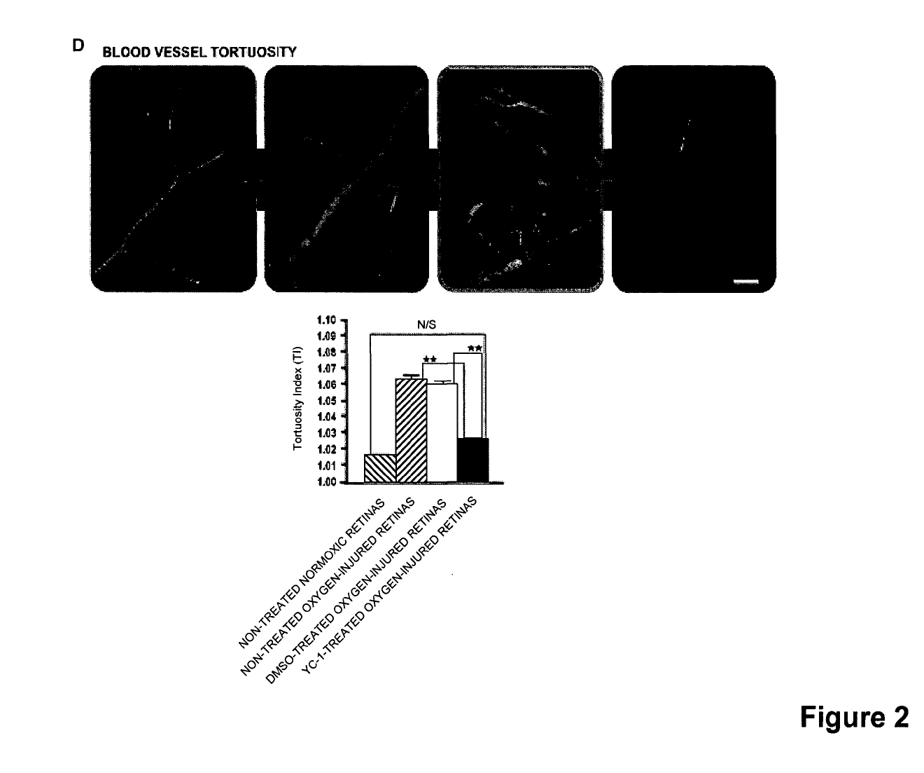

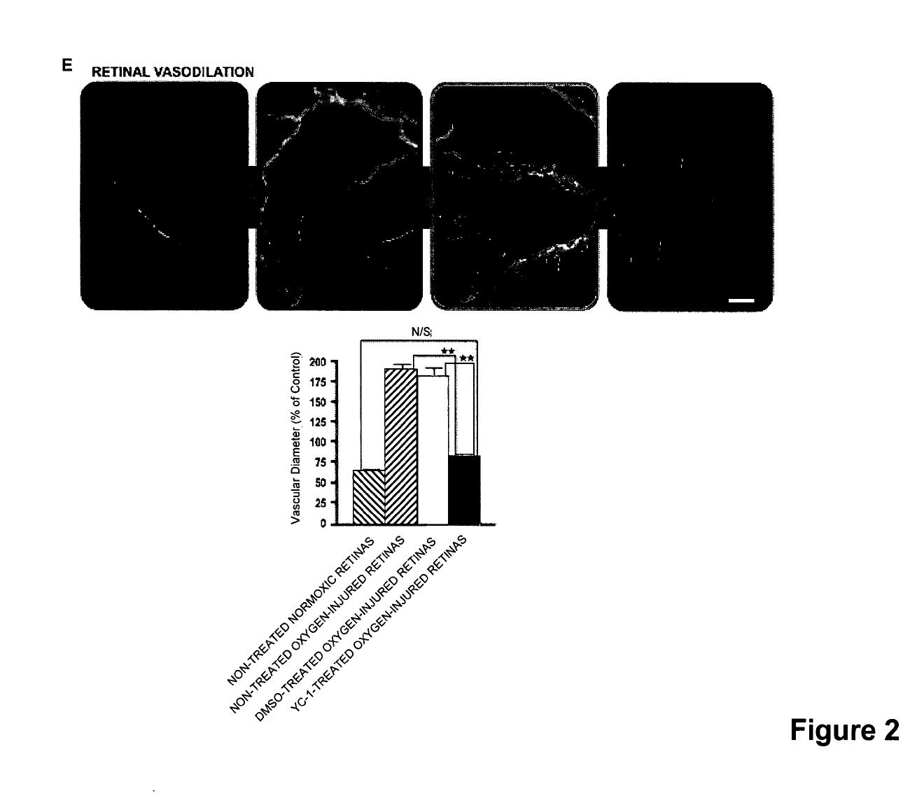

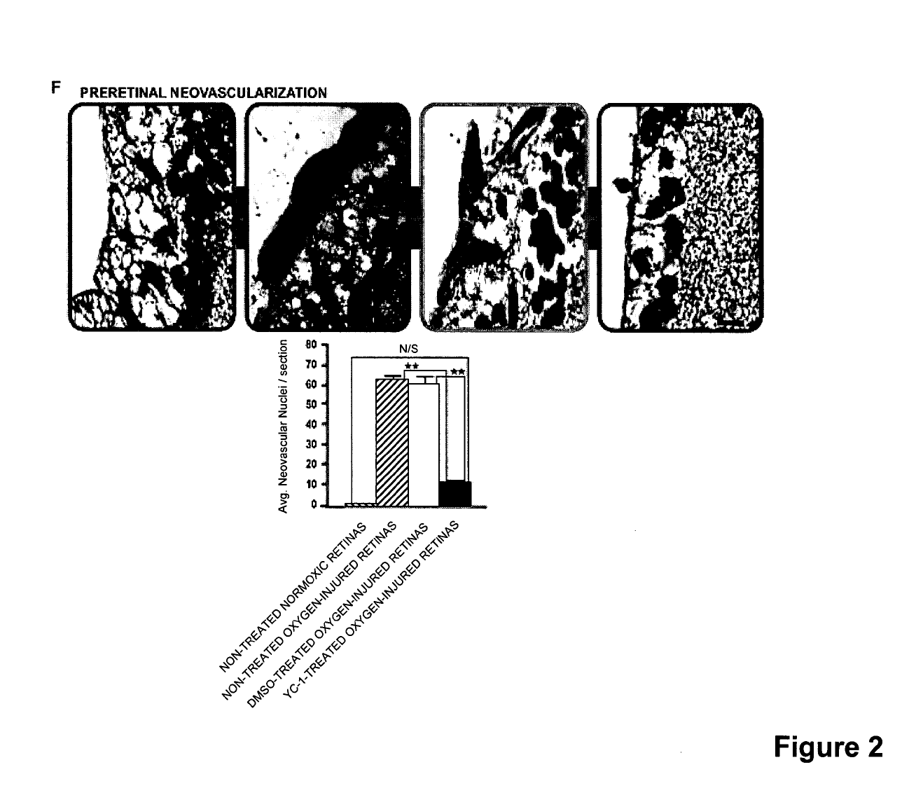

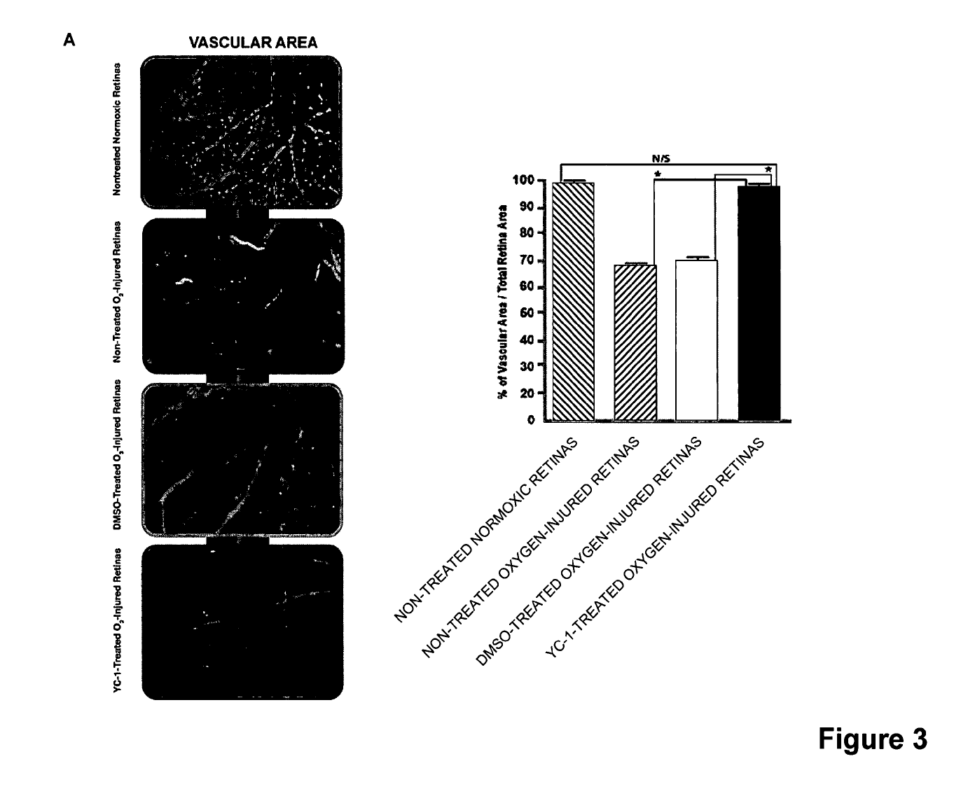

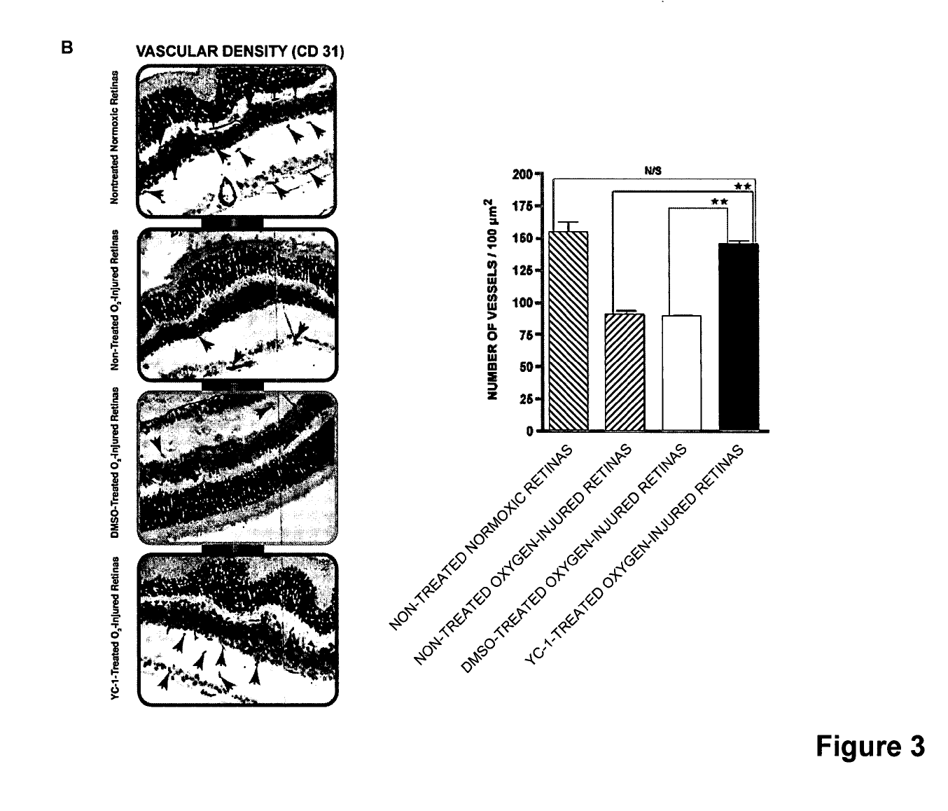

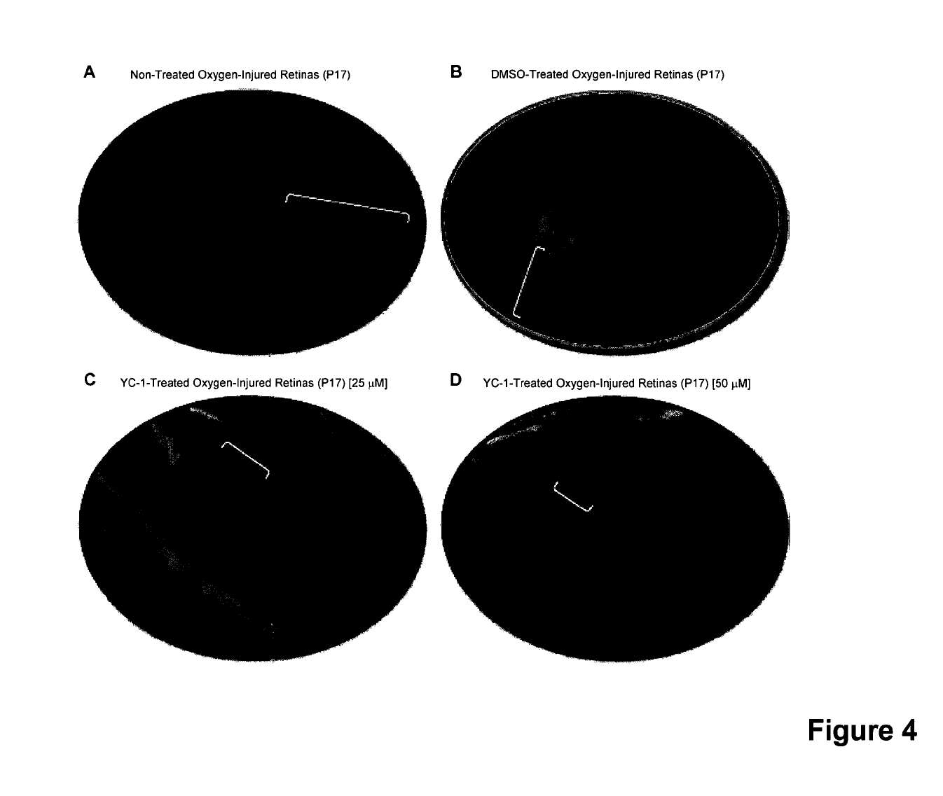

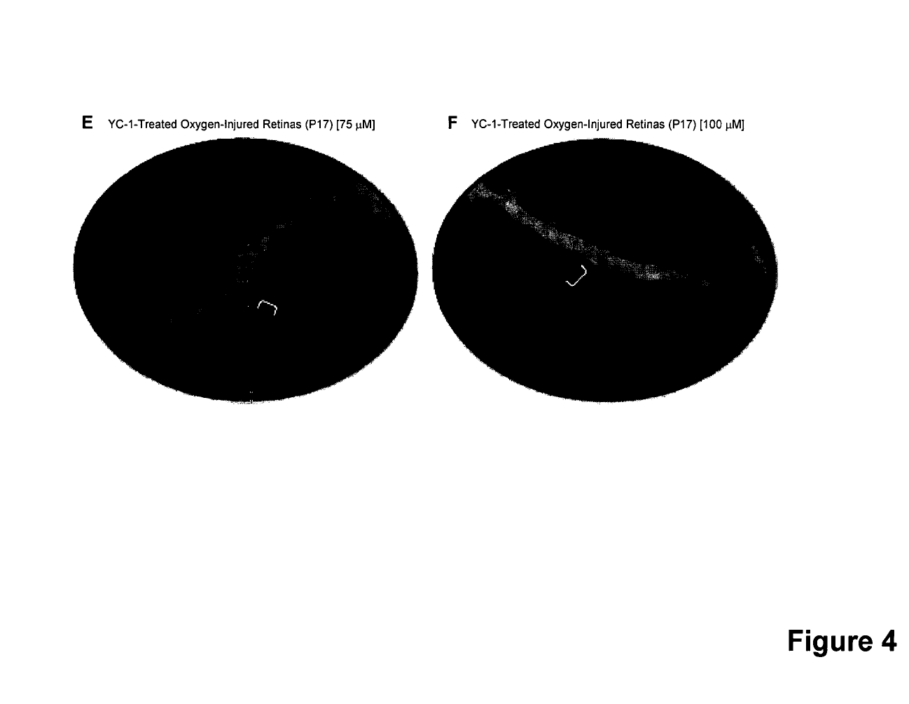

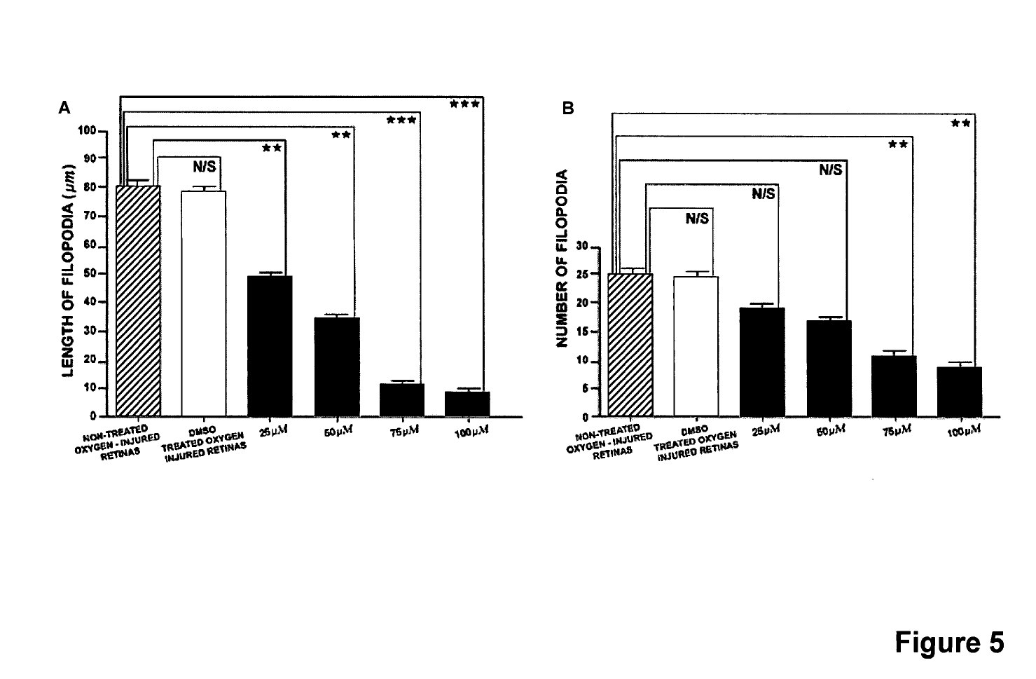

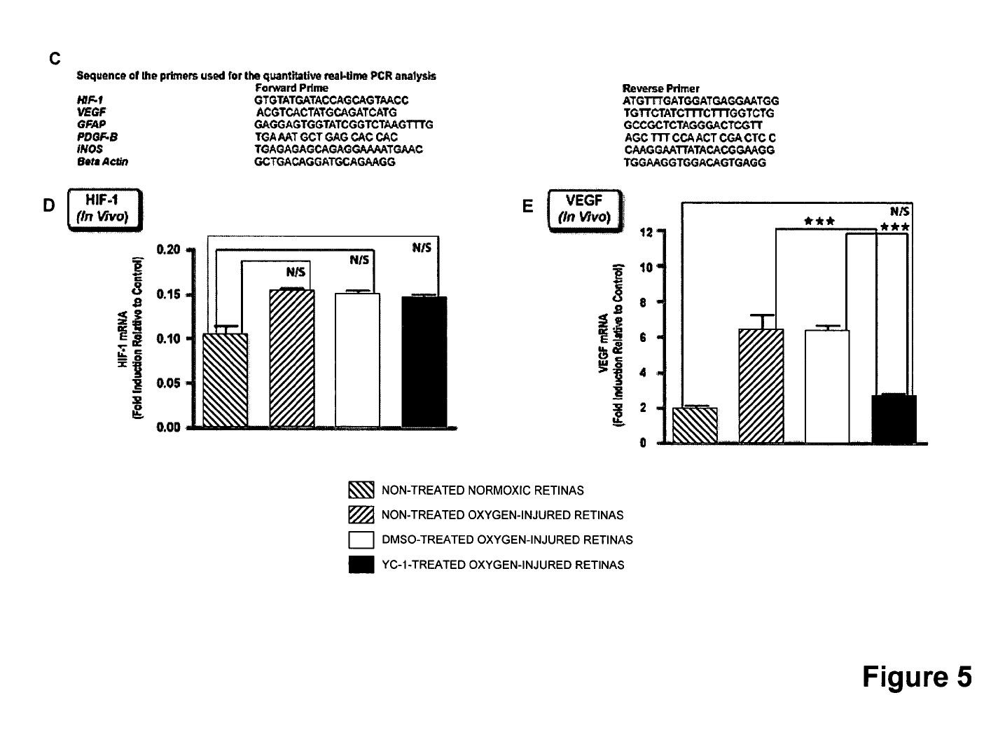

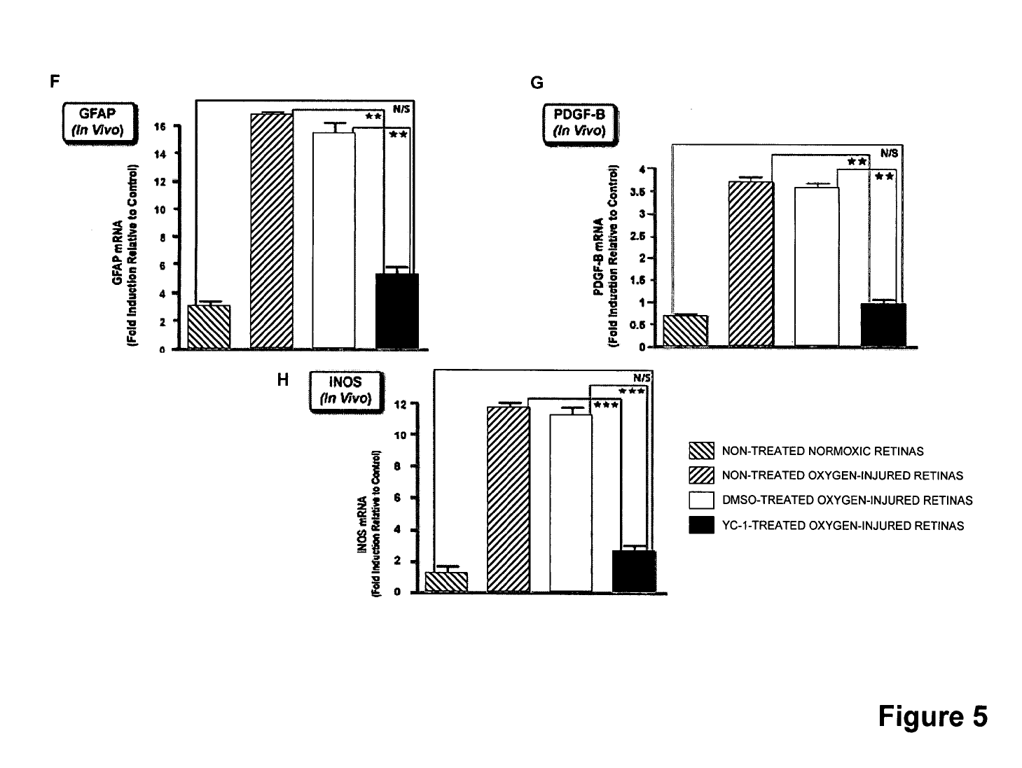

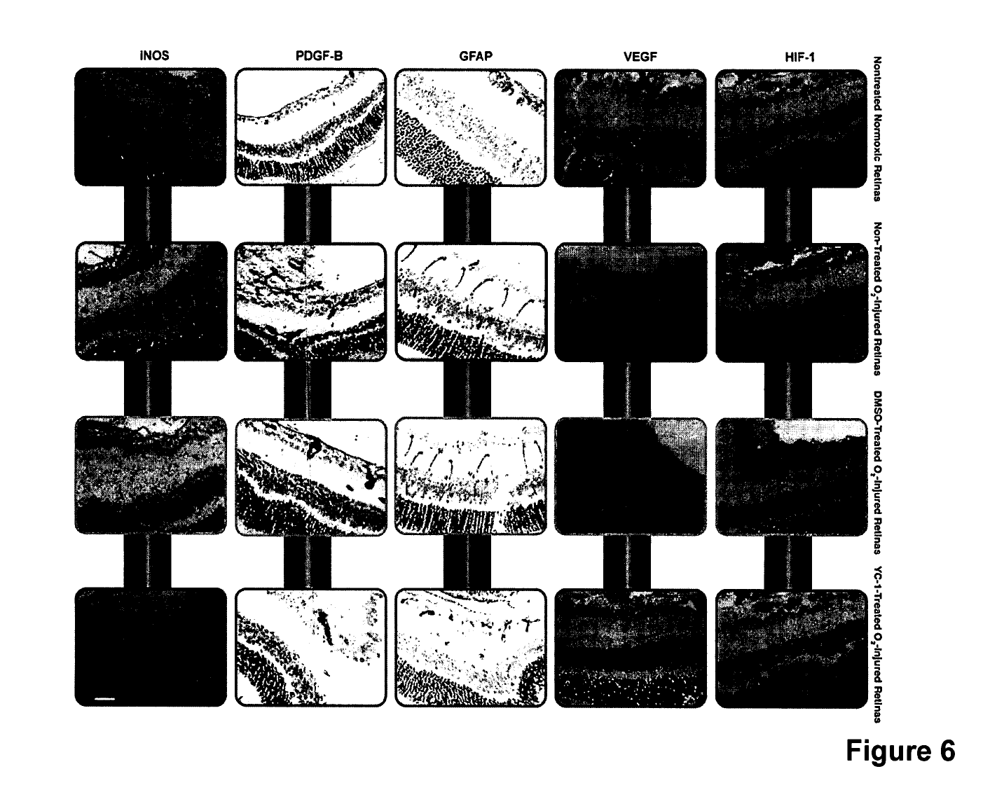

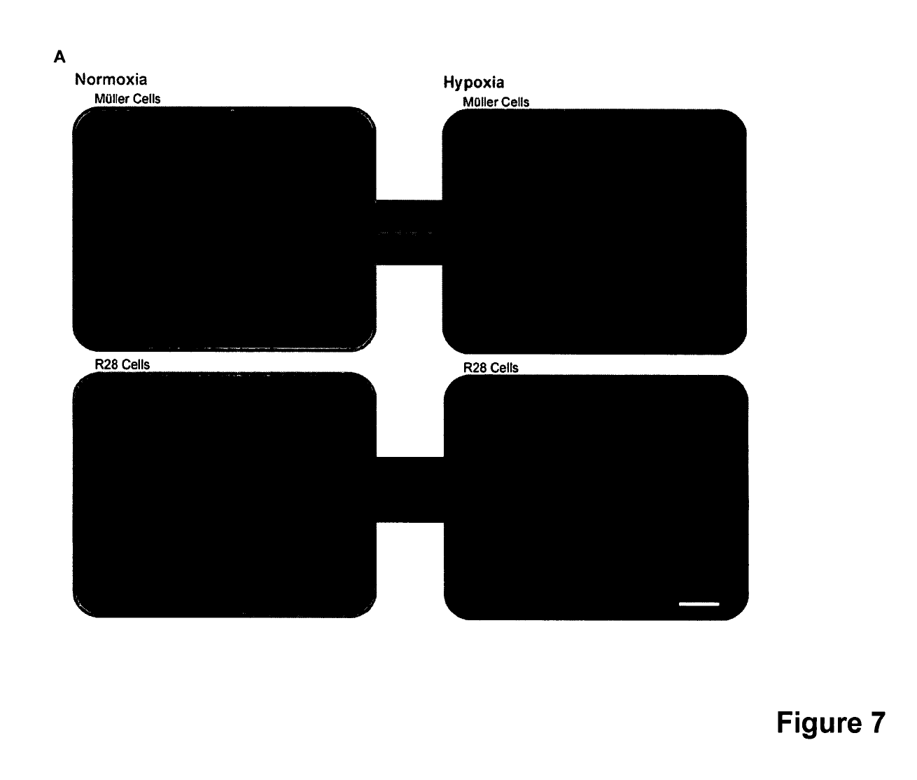

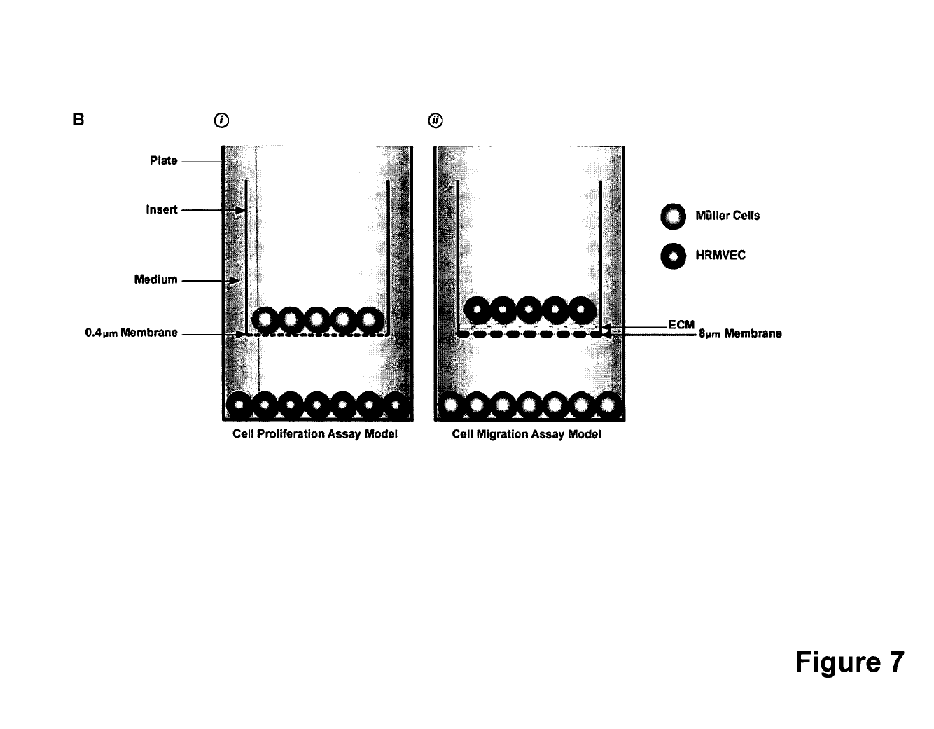

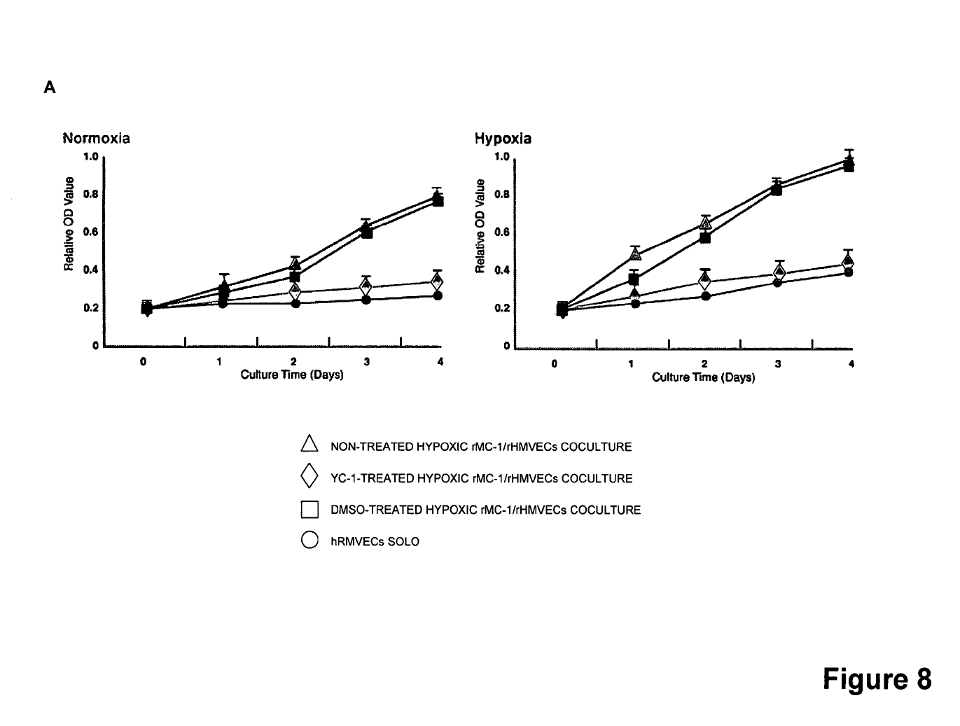

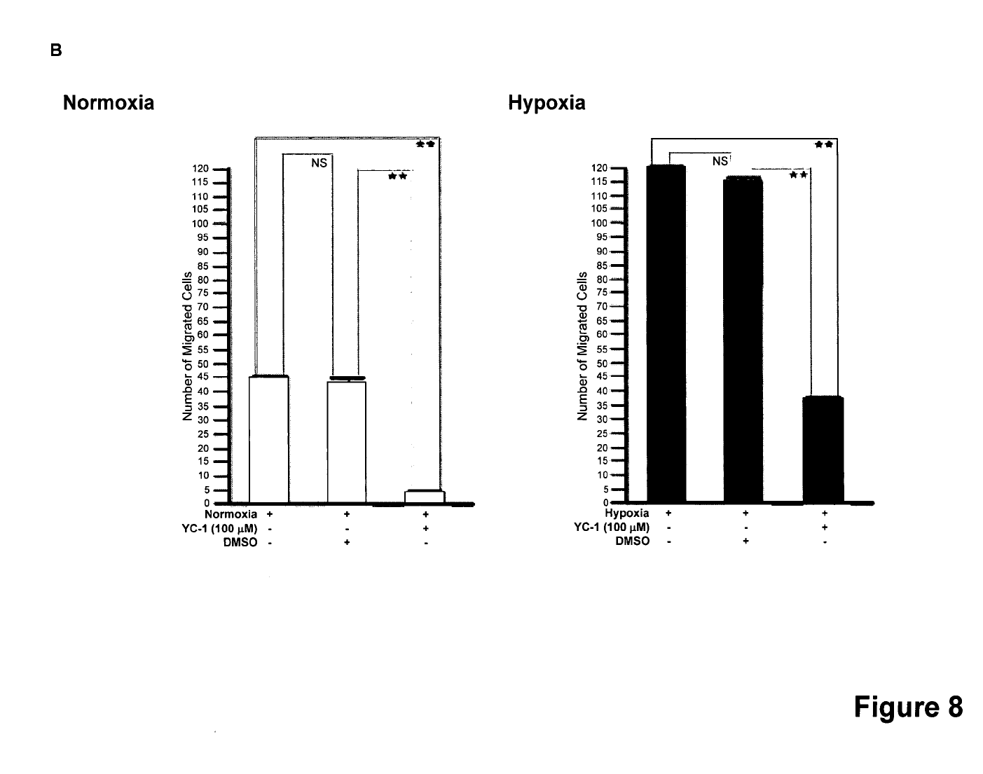

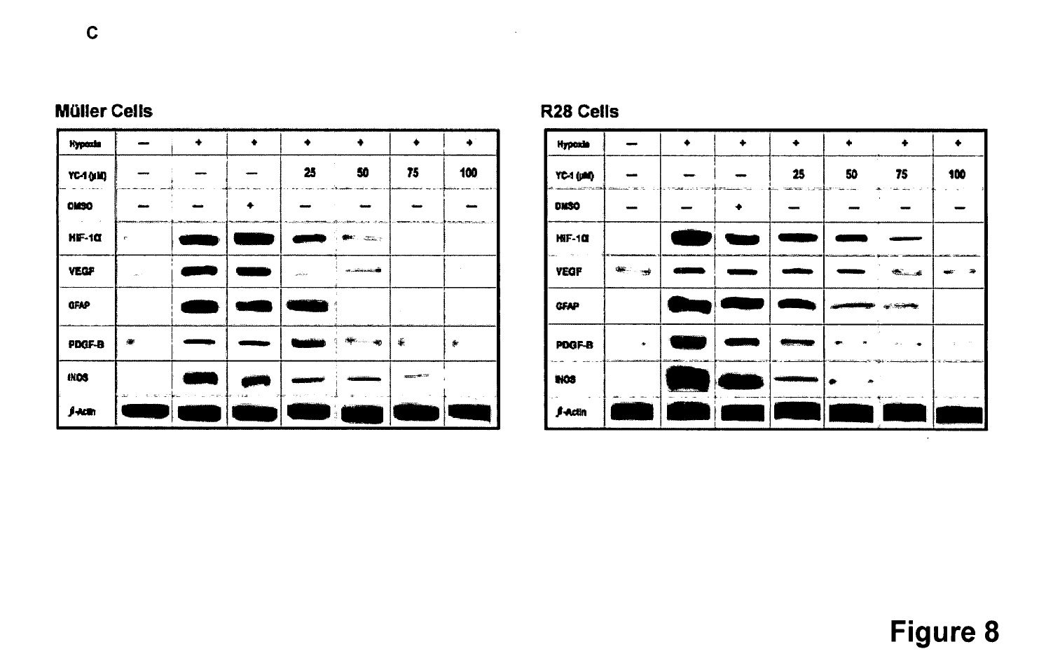

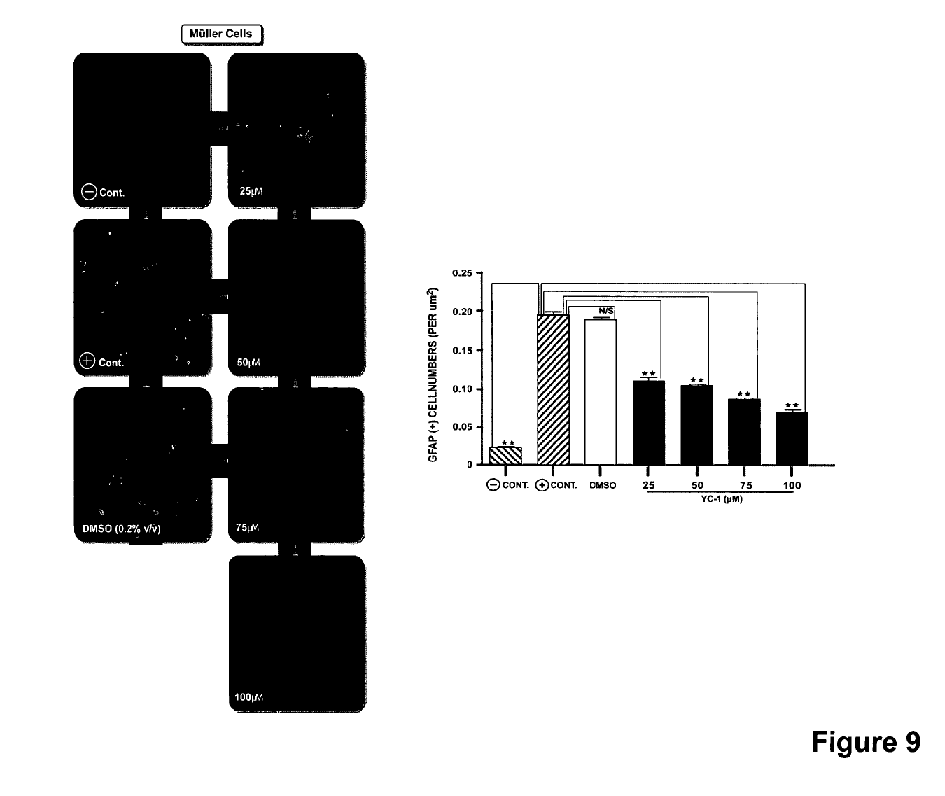

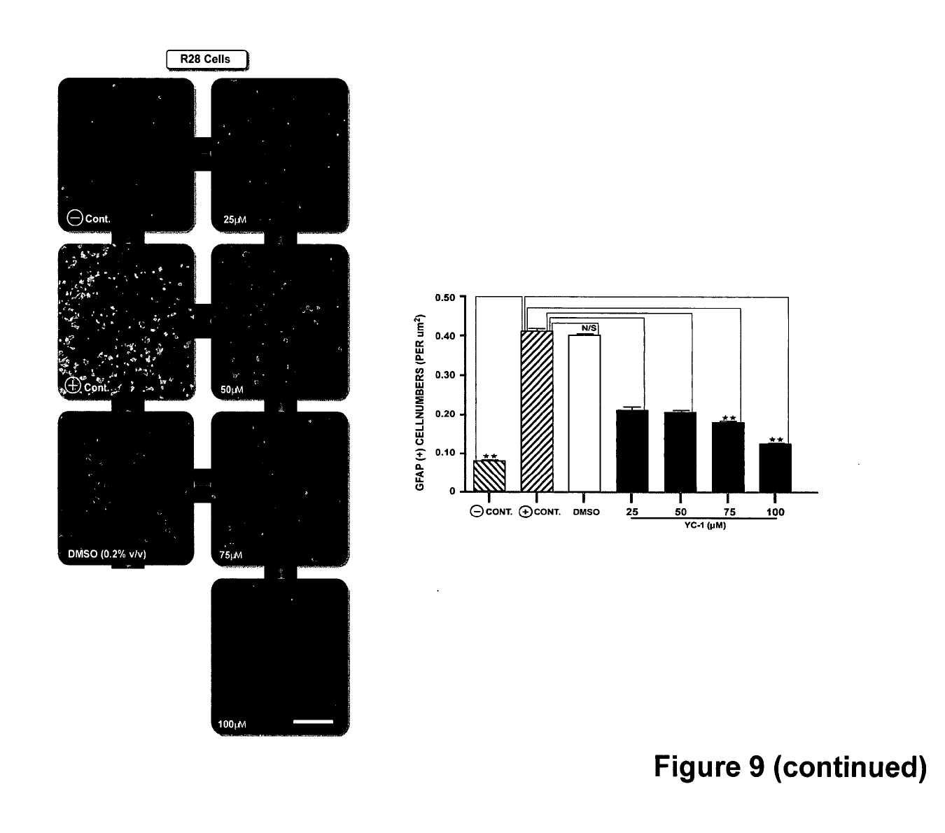

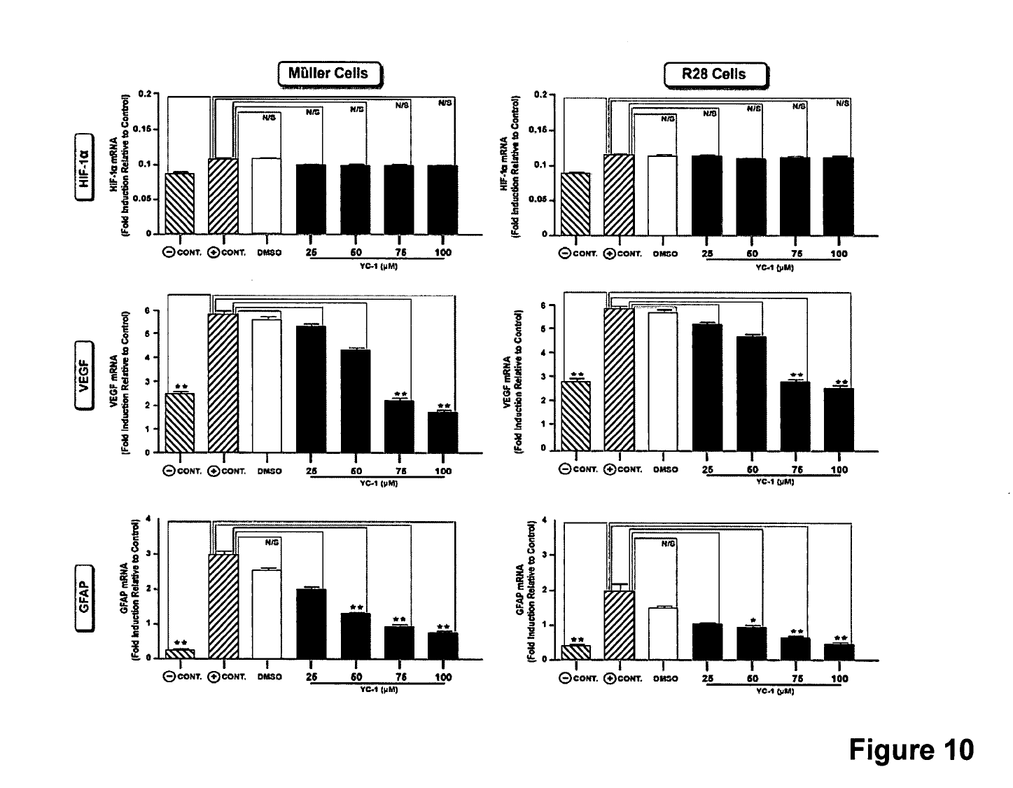

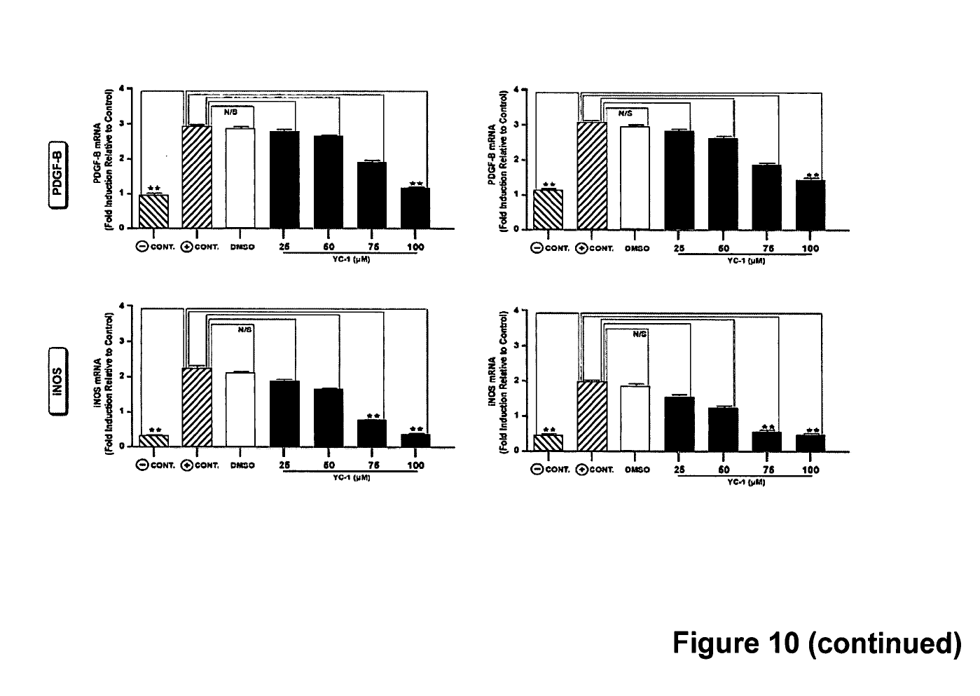

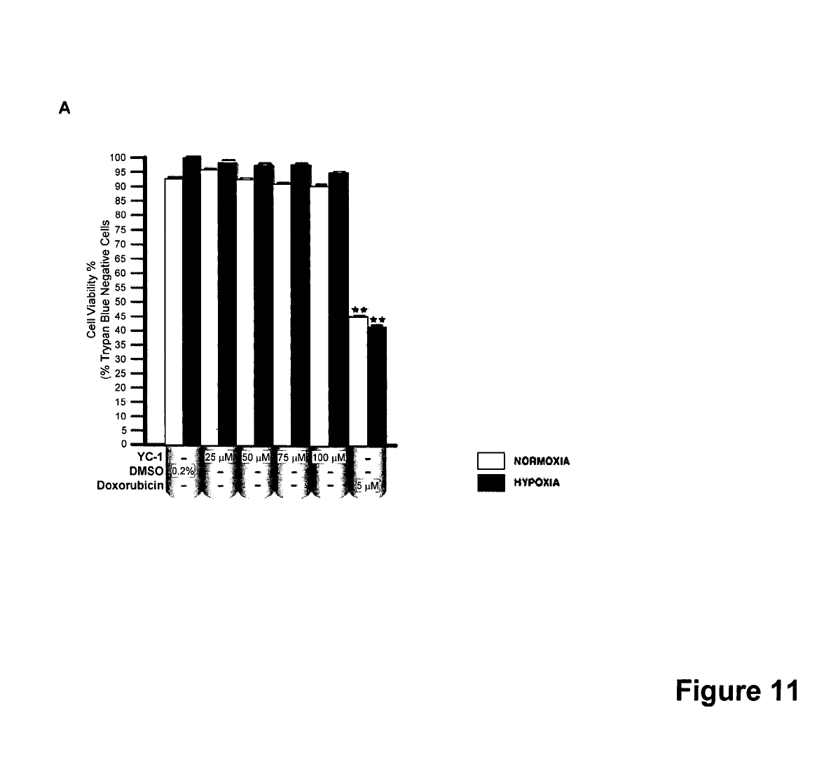

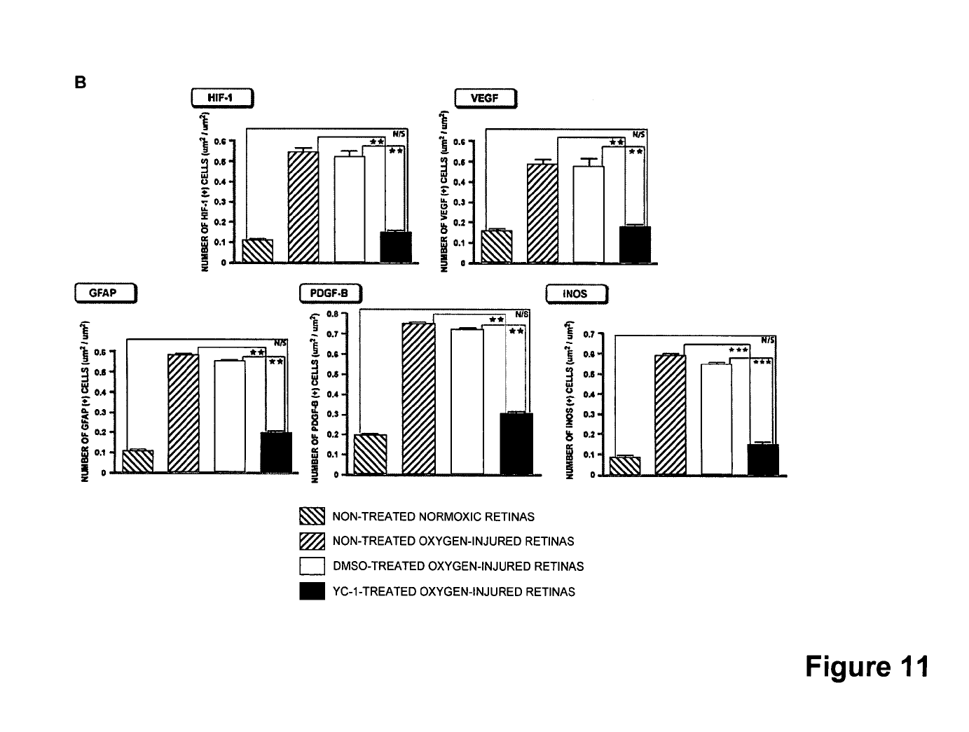

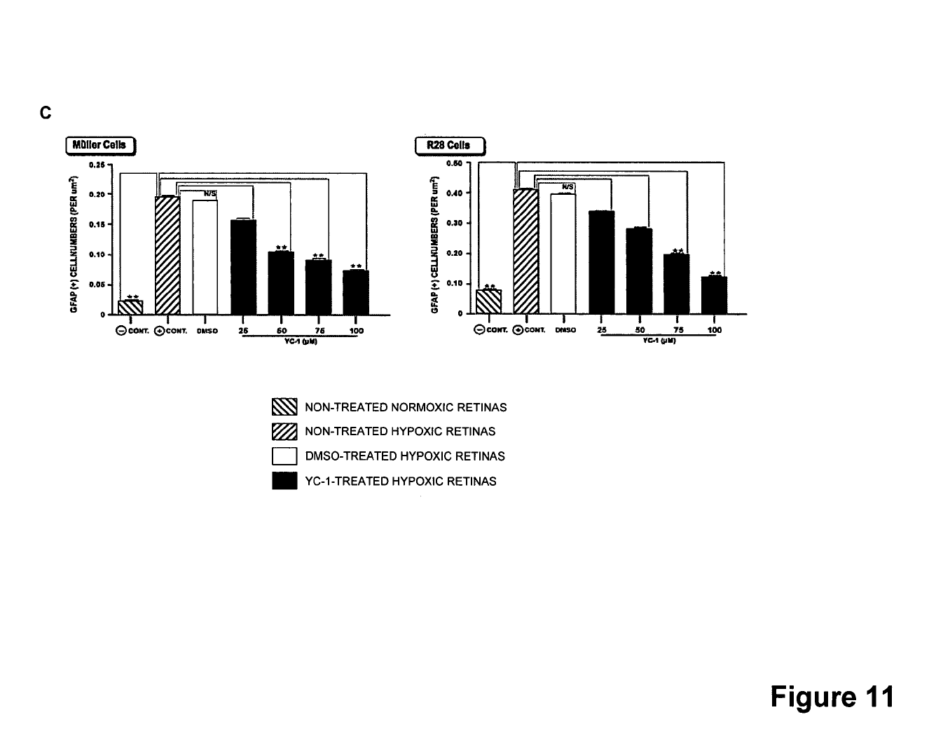

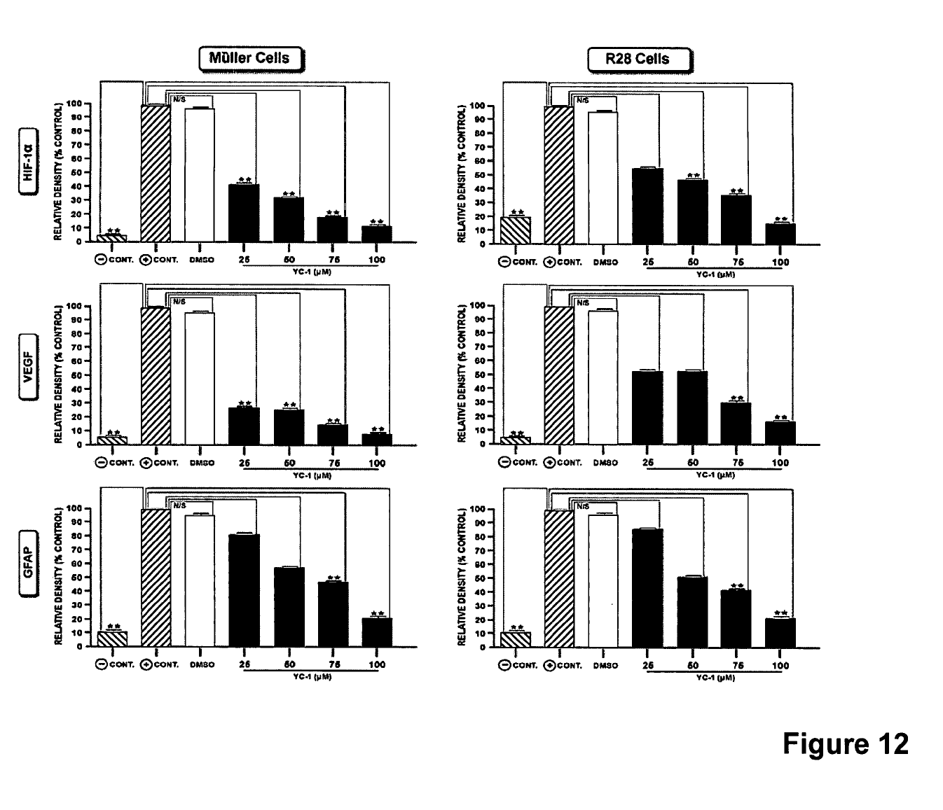

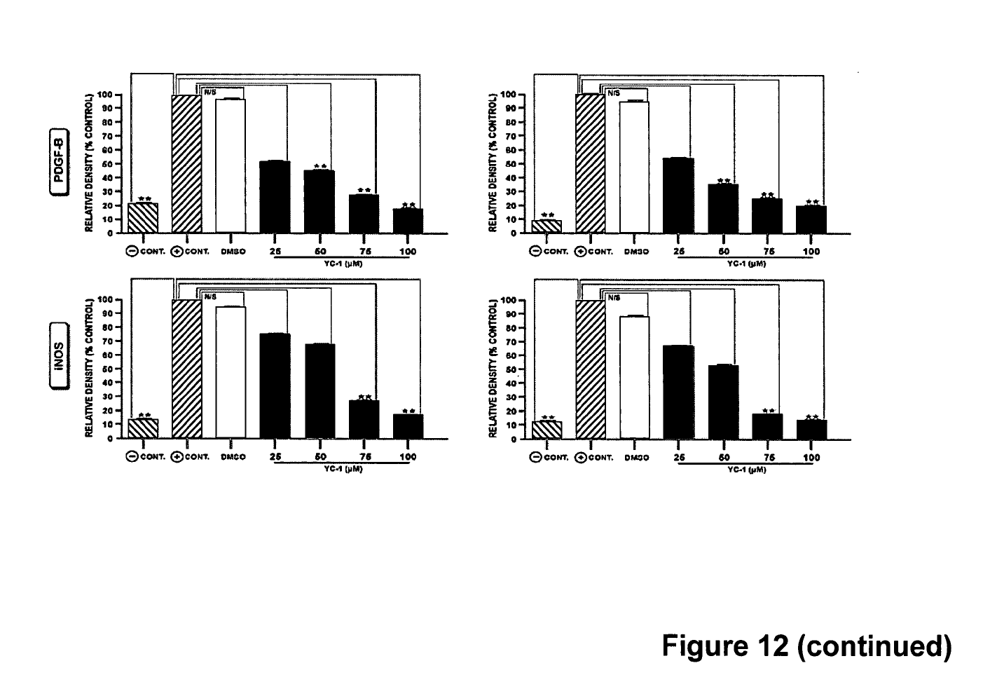

The retinal NV and vascular recovery were studied by comparing the capillary areas as a percentage of the total retinal surface. By P17, retinas from animals that were raised under aerobic conditions exhibited a complete vascular development and remodeling of the retina [FIG. 2A] and all three vascular beds of the retina have been formed and remodeled, resulting in an adult retinal circulation, which is relatively stable thereafter. Furthermore, at P17; the normoxic retinas exhibited the presence of avascularized regions that occupied only [0.002%±0.003] in relation to the size of the entire retina [FIGS. 2A, 2B, Graph in 2B]. However, the ischemic retinas and the DMSO-treated retinas exhibited a significant increase [**P<0.01] of the avascular zones as compared to normoxic controls. These capillary-free zones have arisen within the central retina and appeared as dark regions, which were unperfused by fluorescein [FIG. 2B;]. The total avascularized regions occupied [32%±2.8] and [30%±1.10] in relation to the size of the ischemic retinas and the DMSO-treated retinas, respectively. In the YC-1-treated retinas there was a significant reduction [**P<0.01] in the size of the avascularized regions as compared to DMSO-treated controls. In the YC-1-treated mice, the capillary-free regions occupied only [2.2%±0.1] of the size of the entire retina. Moreover, the normoxic and YC-1-treated retinas exhibited similar patterns in which they exhibited no presence for neovascular vessel tufts [FIG. 2C], and the main vessels were straight and showed no tortuosities [FIG. 2D] or dilation [FIG. 2E]. However, numerous neovascular tufts have protruded from the retina into the vitreous in the ischemic and the DMSO-treated retinas [FIG. 2C]. This was evidenced by formation of more and larger neovascular tufts, thickening of the vessels, and heightened staining of the vasculature with the fluorescent dye in the oxygen exposed animals as compared with mice raised under ambient conditions. High-power magnification of the central capillary-free regions demonstrated extensive “popcorn like” neovascular tufts, which were eventually formed within the superficial capillary network of the neovascularized retinas. Abnormal preretinal neovascular tufts were seen in the mid-periphery, at the interface between the hypovascular central retina and the more vascularized periphery. These neovascular tufts protruded above the ILM of the retina into the vitreous and often persisted until P21 or later. Oxygen treatment significantly increased [**P<0.01] the number of BV tufts from [1±0.01; n=15] in the normoxic retinas to [203±0.73; n=15] in the oxygen-injured retinas [FIG. 2C; Graph in 2C]. DMSO treatment exhibited a similar increasing pattern in the tuft formation [198±0.56; n=15]. While a dual injection of YC-1 resulted in a significant decrease r*P<0.011 of the number of BV tufts/flat mount [3.0±1.1; n=15] [FIG. 2C; Graph (right) in 2C]. In addition, it significantly decreased [**P<0.01] the area of pre-retinal neovascular tufts compared to DMSO-treated group [FIG. 2C; Graph (left) in 2C]. Oxygen injured retinas and DMSO-treated retinas exhibited vasculopathic lesions, which revealed severe degrees of vessel tortuosities [FIG. 2D], dilation of retinal vessels [FIG. 2E], and severe pathological vaso-obliteration that was manifested throughout the entire retinas. Tortuosity indices resulted as follows; normoxic retinas [1.019±0.003]; nontreated ischemic retinas [1.063±0.016]; DMSO-treated retinas [1.059±0.005]; and YC-1-treated retinas [1.026±0.001] [FIG. 2D; Graph in 2D]. Therefore, it appeared that retinal vessels tortuosities were significantly ameliorated by YC-1 treatment [P<0.01]. Whereas no vascular alterations were observed in retinas from eyes that received animals that were raised under ambient conditions; nontreated ischemic retinas and DMSO oxygen-injured retinas exhibited a significant vasodilation [FIG. 2E]. Oxygen injury stimulated vasodilation of major, minor, and capillary vessels. Dual injection of YC-1 on P12 and P15 reduced the dilation of microvessel and significantly ameliorated the retinal vascular changes [**P<0.01]. Quantification of vessel diameters confirmed that YC-1 inhibited oxygen-injury-induced vasodilation and significantly reduced these parameters to values similar to those of the negative controls [FIG. 2E; Graph in 2E]. Moreover, to quantitatively assess the extent of pre-retinal NV; vascular nuclei anterior to the ILM were counted at P17. As expected, in the animals that were grown under ambient conditions, no nuclei were extended into the vitreous [FIG. 2F]. In a marked contrast, all mice, which were exposed to hyperoxia from P7-P 12 and recovered in room air until P17, developed pathologic neovascular tufts extending beyond the ILM into the vitreous. Treatment with double intravitreal injection-regimen of YC-1 [100 μM] resulted in a significant reduction in pre-retinal nuclei, when compared to DMSO-treated retinas. In the non-treated oxygen-injured retinas, the average number of neovascular nuclei was [63±2.8; n=8], whereas the average number of neovascular nuclei in the DMSO-treated retinas [61±2.7; n=8], as compared with (11±1.8; n=8] nuclei in YC-1-treated mouse retinas [FIG. 2F; Graph in 2F]. Additionally, exposure to 75% oxygen led to rapid and profound vasoobliteration thereafter [P12, P15, and P17] as compared with the normoxic group. YC-1 significantly inhibited the retinal vaso-obliteration [FIG. 2A-E] as compared with the obliterated areas of DMSO-treated retinas. YC-1 treatment significantly inhibited of the retinal neovascular response [**P<0.01], preserved and enhanced vasculature, and the retinal vascular tree of these retinas appeared significantly less affected as compared to DMSO treated retinas. No obvious toxic effects on the vasculature were observed in retinas of animals that received YC-1.

### Example 4

### YC-1 Promotes Physiological RV in a Mouse Model of Oxygen Induced Retinopathy (OIR)

Dual intravitreal injections of YC-1 promoted vascular repair and significantly enhanced physiological RV, while the treated retinas appeared fully revascularized as compared with DMSO-treated ischemic retinas [FIG. 3A, n=15]. YC-1 treatment promoted vascular recovery in the ischemic retina, as well as the vascular morphology of YC-1 treated retinas appeared nearly normal. While the vascularized regions occupied [˜99% and ˜97%] of the total size of the retina in the normoxic retinas and the YC-1-treated retinas, respectively; the total vascularized regions occupied only approximately [68% and 70%] in relation to the size of the entire retina of the ischemic retinas and the DMSO-treated retinas, respectively [FIG. 3A; Graph in 3A].

### Example 5

### YC-1-Treated Mice Exhibit Increased Retinal Vascular Density

At P17, retinal sections stained with CD31 from animals that were raised under normoxic conditions had a stable vascular density. Exposure to 75% oxygen led to a significant decrease in the vascular density [**P<0.01] as compared with the normoxic group. YC-1 treatment promoted intraretinal vascular recovery with de novo BV growth and promoted a significant increase [**P<0.01] in the mean vascular density (148±2) as compared to the DMSO-treated mice [88±1] [FIG. 3B; Graph in 3B]. The retinal vascular density in the YC-1 treated mice was comparable to the basal homeostatic level that was exhibited by the non-treated normoxic retinas (154±1).

### Example 6

YC-1 attenuates the filopodial extension in neovascular sprouting from endothelial tip cells in the OIR mouse model

Data from [FIGS. 4A and 4B] demonstrated that retinal NV in the O2-injured and the DMSO treated retinas was associated with the presence of filopodial extensions and the formation of cordlike or tube-like structures. However, YC-1 treatment disturbed the filopodial extension in tip cells [FIGS. 4C-F; and 5A-B]. YC-1 treatment suppressed the development and elongation of neovascular sprouts, and attenuated filopodial extension in sprouting capillaries. YC-1 significantly and dose-dependently reduced the number [FIG. 5A] and the total length [FIG. 5B] of filopodia on endothelial tip cells, as compared to DMSO-treated retinas.

### Example 7

### YC-1 Reverses Retinal Reactive Gliosis In Vivo

Data indicated that there was a significant upregulation in the message and the protein levels of GFAP in the ischemic retina. Since GFAP is a hallmark of RG, this outcome implied the manifestations of RG in response to ischemic injury. There was a significant enhancement of GFAP gene expression levels in the non-treated ischemic retinas as compared with the retinas from animals that were placed under ambient conditions. The effects of DMSO-treatment on the gene expression profiles paralleled those seen in the non-treated ischemic retinas. YC-1 treatment significantly attenuated [**P<0.01] the message levels of GFAP expression [FIG. 5F]. Immunohistochemistry data have demonstrated that the Nontreated O2-Injured Retinas exhibited sparse staining signals for GFAP immunoreactivity, which was mostly associated with astrocytes and Müller cell end feet in the nerve fiber layer [NFL] and GCL [FIG. 6 and Online Resource. 1A]. In addition, these retinas exhibited focal areas of GFAP staining in the INL. In addition, the nontreated O2-injured and the DMSO-treated O2-injured retinas exhibited strong staining of GFAP expression, primarily in the INL, ONL and the GCL of the ischemic retinas. There was strong staining for GFAP immunoreacreactivity in Müller cell processes throughout the retina. In contrast, YC-1-Treated retinas displayed a significant down-regulation in GFAP immunoexpression as compared to DMSO-treated retinas, in addition, GFAP expression was weak “focal”, sporadic and primarily in the GCL and the NFL regions of these retinas.

### Example 8

### YC-1 Downregulates VEGF, PDGF-B, and iNOS Gene Expression Levels in vivo

In order to investigate the mechanisms via which YC-1 reverses retinal RG and NV in the OIR mouse model, we measured the expression of key proteins associated with both; RG and NV. We found that a significant upregulation in the mRNA levels of VEGF, PDGF-B, and iNOS in the ischemic retina as compared to normoxic retinas [FIG. 5E, 5G, 5H]. Treatment of the animals with 100 μM YC-1 significantly attenuated the message levels of VEGF [**P<0.01], PDGF-B [**P<0.01], and iNOS [***P<0.001] as compared to DMSO-treated retinas. However, there were no significant differences between HIF-1α message levels under normoxic or ischemic microenvironments. Treatment with DMSO or YC-1 did not have any influence on HIF-1α message levels in retinal tissue [FIG. 5D].

### Example 9

### Immunohistochemical Localization of HIF-1, VEGF, PDGF-B and iNOS

Non-Treated Normoxic Retinas [FIG. 6 and Supplementary. 1B] expressed detectable basal levels of HIF-1α and VEGF in the INL, GCL, and NFL. PDGF-B was predominantly localized in the OPL, INL, GCL, and NFL. However, iNOS immunostaining revealed very weak but detectable immunoreactivity that was primarily expressed in the GCL. In the Non-Treated Ischemic Retinas exhibited the presence of patchy strong HIF-1α overexpression, primarily in the INL, IPL, GCL, and NFL. There was marked elevation of VEGF expression in the INL, GCL, and NFL. The pattern of PDGF-B immunohistolocalization did not alter from the normoxic retinas. However, ganglion cells were particularly prominent, as well as significant upregulation in the positive immunoreactivities of PDGF-B was present within the inner segments of the photoreceptor cells/INL. The immunoreactivity was patchy with strong PDGF-B overexpression and primarily augmented in the INL and GCL, as compared to normoxic retinas and the YC-1-treated retinas. Furthermore, there was a significant increase in the level of iNOS expression in the outer plexiform layer.[OPL], INL, IPL, GCL and NFL.

In the DMSO-Treated Retinas, the staining intensity of HIF-1 and VEGF were strong and significantly elevated in the INL, GCL, and NFL, when compared to YC-1-treated retinas.

The immunohistolocalization pattern of PDGF-B was identical to the pattern that was shown in the ischemic controls. The immunolabeling of iNOS was markedly increased in the OPL, INL, IPL, GCL, and NFL. In contrast, the YC-1-treated retinas displayed a significant inhibition in HIF-1 and VEGF immunoexpressions. While HIF-1 staining was primarily located in the GCL; VEGF staining was weak “focal”, sporadic and primarily in the INL, GCL, and NFL regions. PDGF-B immunoreactivity was significantly inhibited compared with DMSO-treated retinas, and the immunostaining was primarily localized in the INL and GCL.

iNOS immunoreactivities were detectable but moderate, and significantly down-regulated compared to DMSO-treated retinas. Immunostaining was primarily localized in the GCL with weak staining in the INL.

### Example 10

### Hypoxia Alters the Morphology of rMC-1 and R28Cells

In view of the fact that Müller cells processes, which span the entire retina and extend from the INL to the OLM, the possibility exists that YC-1 maybe acting directly on retinal glial cells. Hence we have decided to select two glial cell lines; Müller cells [rMC-1] and retinal neuoroglial cells [R28 cells]. The morphology and the growth pattern of rMC-1 and R28 cells were examined under light microscopy. Under normoxic conditions, the R28 cells adopt a neuronal morphology, with neurite-like projections, while rMC-1 cells are large and flat cells [FIG. 7A]. Hypoxic exposure for 72 hours induced apparent hypertrophic morphological changes in rMC-1 and R28 cells. Most of the hypoxic cells exhibited moderate to severe degrees of hypertrophy.

### Example 11

Inhibition of ECs Proliferation and Migration in the Coculture System via the Anti-angiogenic Effects of YC-1 on Müller Cells

The proliferation assay in a coculture system model [FIG. 7B] demonstrates that rMC-1 cells-hRMVECs coculture significantly increased hRMVECs proliferation compared to solo hRMVECs culture [FIG. 8A] under normoxia and hypoxia. Data have indicated that coculture under hypoxic conditions had a synergistic effect. Although there was insignificant difference in the hRMVECs proliferation while being cocultured with nontreated rMC-1 cells under normoxic or hypoxic conditions; the proliferation of hRMVECs was significantly suppressed when rMC-1 cells were treated with YC-1, under normoxic and hypoxic conditions.

Furthermore, migration assay in a coculture system model has shown that hRMVECs were found to extend through 8.0 mm Transwell pores in a transmigration assay with Müller cells grown in the well [FIG. 7B]. Coculture of hRMVECs with Müller cells under hypoxia resulted in a significant increase in hRMVECs migratory activity over the levels of [rMC-1 cells/normoxia group]. Whereas the rMC-1 cells-induced hRMVECs migration was significantly attenuated by YC-1 treatment under normoxic [rMC-1 cells/hRMVECs (normoxia group)] and hypoxic conditions [rMC-1 cells/hRMVECs (hypoxia group)] [FIG. 8B].

### Example 12

### YC-1 Restrains Hypoxia-Induced Upregulation of VEGF, PDGF-B and iNOS mRNA Levels in vitro

Real time RT-PCR data have demonstrated that after 48 hours of hypoxic exposure, the levels of VEGF, PDGF-B, and iNOS mRNA expression were significantly increased over the normoxic control, which displayed low gene expression levels [FIG. 10]. However, there were no differences of HIF-1α message levels between normoxic and hypoxic cells; or between pre- and post-YC-1 treatment. Treatment of both cell lines YC-1 [25-100 μM] resulted in significant dose-dependent attenuations in the message levels of VEGF, PDGF-B and iNOS expressions, when compared with DMSO-treated hypoxic cells.

### Example 13

### YC-1 Inhibits HIF-1, VEGF, PDGF-B and iNOS Protein Levels in Glial Cells

In order to investigate the mechanisms via which YC-1 reverses retinal RG and NV in the cell culture models, we quantified the expression of key proteins associated with both; RG and NV. Western immunoblot analysis indicated that rMC-1 and R28 cells cultured under normoxia exhibited low immunoreactivity signals of HIF-1, VEGF, PDGF-B, and iNOS protein expression, while this signal was overexpressed after 48 hours of hypoxic exposure [FIG. 8C]. There was a significant increase in HIF-1, VEGF, PDGF-B, and iNOS protein expression, as measured by densitometry [FIG. 12], compared to normoxia. In both cell types and under hypoxia, YC-1 inhibited the hypoxia-induced upregulation of HIF-1, VEGF, PDGF-B, and iNOS protein levels in a dose-dependent manner, compared with DMSO-treated hypoxic cells.

### Example 14

### YC-1 Reverses Retinal Reactive Gliosis in vitro

Western immunoblot analysis has indicated that rMC-1 and R28 cells cultured under normoxia exhibited low immunoreactivity signals of GFAP protein expression, while this signal was overexpressed after 48 hours of hypoxic exposure [FIG. 8C]. In the DMSO-treated hypoxic cells, GFAP protein expression remained relatively stable, when compared with nontreated hypoxic cells. This increase in GFAP expression was measured by densitometry [FIG. 12]. In both cell types and under hypoxic conditions; YC-1 treatment inhibited the hypoxia-induced GFAP protein levels in a dose-dependent manner, compared with non-treated hypoxic cells. Immunofluorescence staining of GFAP demonstrated that non-treated rMC-1 and R28 cells cultured under hypoxia displayed enriched GFAP protein fluorescence immunoreactivity, with strong cytoplasmic staining of both cell types [FIG. 9; positive control]. Hypoxic cells exhibited significant increase in GFAP protein levels, as compared with normoxic cells, which exhibited limited areas of very weak GFAP staining [FIG. 9; negative control].

Furthermore, there was a strong positive GFAP staining signal deposited over the cytoplasms of the DMSO-treated cells cultured for 48 hours under hypoxia [FIG. 9; DMSO]. rMC-1 cells that were treated with 25 μM YC-1 displayed the presence of cytoplasmic localization but then with weaker equivocal staining intensity. Whereas, a stronger diffuse cytoplasmic GFAP staining was observed in R28 cells. Treatment of rMC-1 and R28 cells with 25 μM YC-1 under hypoxia for 48 hours displayed a remarkable inhibition, compared to DMSO-treated hypoxic controls. At [50 and 75 μM], YC-1 had significant inhibitory effects on GFAP protein expression in both cell types, as compared to hypoxic controls. At 100 μM YC-1, there were few stained regions that were still detected in the cytoplasm of YC-1-treated cells.

Data indicated that YC-1 significantly reduced GFAP protein levels in R28 and rMC-1 cells under hypoxia and in a dose-dependent fashion [**P<0.01] [FIG. 11C]. The Real time RT-PCR data have revealed that post hypoxic exposure, the level of GFAP mRNA expression was significantly increased over the normoxic control, which displayed a basal expression levels [FIG. 10]. The effects of DMSO-treatment on the gene expression patterns in both cell types paralleled those seen in the non-treated ischemic retinas. Treatment of r-MC1 and R28 cells with 25-100 μM YC-1 resulted in significant dose-dependent attenuations in the message levels of GFAP expression, when compared with DMSO-treated hypoxic cells. Data were normalized to β-actin mRNA levels.

### REFERENCES

[1] Amin R H, F. R., Kennedy A, Eliott D, Puklin J E, Abrams G W (1997) Vascular endothelial growth factor is present in glial cells of the retina and optic nerve of human subjects with nonproliferative diabetic retinopathy. Invest Ophthalmol V is Sci. 36-47.
[2] Bergers G, S. S., Meyer-Morse N, Bergsland E, Hanahan D (2003) Benefits of targeting both pericytes and endothelial cells in the tumor vasculature with kinase inhibitors. J Clin Invest. 1287-1295.
[3] Bressler J, G. G., Levitov C, Hjelmeland L (1985) Chemotaxis of rat brain astrocytes to platelet-derived growth factor. Brain Res. 249-254.
[4] Bringmann A, P. T., Grosche J, Francke M, Wiedemann P, Skatchkov S N, Osborne N N, Reichenbach A (2006) Müller cells in the healthy and diseased retina. Prog Retin Eye Res. 397-424.
[5] Brooks S E, G. X., Samuel S, Marcus DM (2001) Reduced severity of oxygen-induced retinopathy in eNOS-deficient mice. Invest Ophthalmol V is Sci. 42: 222-228.
[6] Brosnan C F, B. L., Raine C S, Dickson D W, Casadevall A, Lee S C (1994) Reactive nitrogen intermediates in human neuropathology: an overview. Dev Neurosci. 152-161.
[7] Büchi ER, K. A., Tso M O (1996) Retinopathy in diabetic hypertensive monkeys: a pathologic study. Graefes Arch Clin Exp Ophthalmol. 388-398.
[8] Campochiaro, P. (2010) Molecular Targets for Retinal Diseases: Hypoxia-regulated genes play an important role in vascular permeability and neovascularization. Retina Today. 4-7.
[9] Campochiaro P A, G. B. (1985) Platelet-derived growth factor is chemotactic for human retinal pigment epithelial cells. Arch Ophthalmol. 576-579.
[10] Campochiaro P A, H. S. (2003) Ocular neovascularization: a valuable model system. Oncogene. 6537-6548.
[11] Chen F, M. M., Zhang Q, Wei N, Chen K, Xu H, Yuan J, Zhou Y, Lang H, Yu X, Wang B, Wang J, Tang Y, Chang H (2010) Taurine buffers glutamate homeostasis in retinal cells in vitro under hypoxic conditions. Ophthalmic Res. 105-112.
[12] Chiou S H, C. C., Hsu W M, Kao C L, Liu J H, Chen W L, Tsai D C, Wu C C, Chou C K (2001) Elevated nitric oxide level in aqueous humor of patients with acute angle-closure glaucoma. Ophthalmologica. 215: 113-116.
[13] Corbett, J. A., Tilton, R. G., Chang, K., Hasan, K. S., Ido, Y., Wang, J. L., Sweetland, M. A., Lancaster, J., Jr., Williamson, J. R. and McDaniel, M. L (1992) Aminoguanidine, a novel inhibitor of nitric oxide formation, prevents diabetic vascular dysfunction. Diabetes. 552-556.
[14] Cox O T, S. D., Stitt A W, Gardiner T A (2003) Sources of PDGF expression in murine retina and the effect of short-term diabetes. Mol. Vis. 665-672.
[15] Cunha-Vaz J, F. d. A. J., Campos A J (1975) Early breakdown of the blood-retinal barrier in diabetes. Br J. Ophthalmol. 649-656.
[16] DeNiro M, A.-H. A., Al-Mohanna F H, Alsmadi O, Al-Mohanna F A (2010) Pleiotropic effects of YC-1 selectively inhibit pathological retinal neovascularization and promote physiological revascularization in a mouse model of oxygen-induced retinopathy. Mol. Pharmacol. 348-367.
[17] DeNiro M, A.-M. F., Al-Halafi A, Alsmadi O, Al-Mohanna F (2009) Pleiotropic Effects of YC-1 Attenuates Pathological Retinal Neovascularization. Invest Ophthalmol V is Sci; ARVO E-Abstract#41.2480.
[18] DeNiro M, A. O., Al-Mohanna F (2009) Modulating the Hypoxia-Inducible Factor Signaling Pathway as a Therapeutic Modality to Regulate Retinal Angiogenesis. Experimental Eye Research.
[19] Du Y, S. M., Miller C M, Kern T S (2002) Diabetes-induced nitrative stress in the retina, and correction by aminoguanidine. J. Neurochem. 771-779.
[20] Eisenfeld A J, B.-M. A., Sarthy P V (1984) Müller cell expression of glial fibrillary acidic protein after genetic and experimental photoreceptor degeneration in the rat retina. Invest Ophthalmol V is Sci. 1321-1328.
[21] Ellis E A, S, N., Caballero S, Guthrie S M, Mames R N, Grant M B (2005) Nitric oxide synthases modulate progenitor and resident endothelial cell behavior in galactosemia. Antioxid Redox. 1413-1422.
[22] Erickson P A, F. S., Guerin C J, Anderson D H, Kaska D D (1987) Glial fibrillary acidic protein in Muller cells after retinal detachment. Exp Eye Res. 37-48.
[23] Friebe A, K. D. (2003) Regulation of nitric oxide-sensitive guanylyl cyclase. Circ Res. 96-105.
[24] Friedl P, H. Y., Tusch M (2004) Collective cell migration in morphogenesis and cancer. Int J Dev Biol. 441-449.
[25] Gelman R, M.-P. M., Vanderveen D K, Moskowitz A, Fulton A B (2005) Diagnosis of plus disease in retinopathy of prematurity using Retinal Image multiScale Analysis. Invest Ophthalmol V is Sci. 4734-4738.
[26] Gerhardt H, G. M., Fruttiger M, et al (2003) VEGF guides angiogenic sprouting utilizing endothelial tip cell filopodia. J. Cell Biol. 1163-1177.
[27] Gu X, S. S., El-Shabrawey M, Caldwell R B, Bartoli M, Marcus D M, Brooks S E (2002) Effects of sustained hyperoxia on revascularization in experimental retinopathy of prematurity. Invest Ophthalmol V is Sci. 496-502.
[28] Hayashi A, K. B., Imai K, de Juan E Jr (1996) ncrease of protein tyrosine phosphorylation in rat retina after ischemia-reperfusion injury. Invest Ophthalmol V is Sci. 2146-2156.
[29] He T, X. Y., Zhao X H, Ai M (2007) Interaction between iNOS and COX-2 in hypoxia-induced retinal neovascularization in mice. Arch Med. Res. 807-815.
[30] Heldin CH, W. B. (1999) Mechanism of action and in vivo role of platelet-derived growth factor. Physiol Rev. 1283-1316.
[31] Hellstrom A, S. E., Carlsson B, Niklasson A, Albertsson-Wikland K (1999) Reduced retinal vascularization in children with growth hormone deficiency. J Clin Endocrinol Metab. 795-798.
[32] Hsiao G, H. H., Fong T H, et al. (2004) Inhibitory mechanisms of YC-1 and PMC in the induction of iNOS expression by lipoteichoic acid in RAW 264.7 macrophages. Biochem Pharmacol 67: 1411-1419.
[33] Jing R, W. U., Goodwill W, Li L, Pan Y, Pekny M, Skalli O (2007) Synemin is expressed in reactive astrocytes in neurotrauma and interacts differentially with vimentin and GFAP intermediate filament networks. J Cell Sci. 1267-1277.
[34] Kaur C, S. V., Yong Z, Lu J, Foulds W S, Ling E A (2007) Blood-retinal barrier disruption and ultrastructural changes in the hypoxic retina in adult rats: the beneficial effect of melatonin administration. J. Pathol. 429-439.
[35] Kawasaki A, O. Y., Barnstable C J (2000) Müller cell protection of rat retinal ganglion cells from glutamate and nitric oxide neurotoxicity. Invest Ophthalmol V is Sci. 3444-3450.
[36] Kim, I. B., Kim, K. Y., Joo, C. K., Lee, M. Y., Oh, S. J., Chung, J. W., Chun, M. H (1998) Reaction of Mu\ddot{}ller cells after increased intraocular pressure in the rat retina. Exp. Brain Res. 419-424.
[37] Klein R, K. B., Moss S E, Davis M D and DeMets D L (1984) The Wisconsin epidemiologic study of diabetic retinopathy. II. Prevalence and risk of diabetic retinopathy when age at diagnosis is less than 30 years. Arch Ophthalmol. 520-526.
[38] Ko F N, W. C., Kuo S C, Lee F Y, Teng C M (1994) YC-1, a novel activator of platelet guanylate cyclase. Blood. 4226-4233.
[39] Kroll J, W. J. (1998) VEGF-A induces expression of eNOS and iNOS in endothelial cells via VEGF receptor-2 (KDR). Biochem Biophys Res Commun. 743-746.
[40] Krupinski J, I. R., Bujny T, Slevin M, Kumar P, Kumar S, Kaluza J (1997) A putative role for platelet-derived growth factor in angiogenesis and neuroprotection after ischemic stroke in humans. Stroke. 564-573.
[41] Lakshminarayanan S, A. D., Gardner T W, Tarbell J M (2000) Effect of VEGF on retinal microvascular endothelial hydraulic conductivity: the role of NO. Invest Ophthalmol V is Sci. 4256-4261.
[42] Lefrançois T, F. C., Peschanski M & Tardy M (1997) Neurite outgrowth associated with astroglial phenotypic changesinduced by antisense glial fibrillary acidic protein (GFAP) mRNA in injured neuronastrocyte cocultures. Journal of Neuroscience. 4121-4128.
[43] Lieth E, B. A., Xu B, Dice C, Ratz M J, Tanaser D, Strother J M et al (1998) Glial reactivity and impaired glutamate metabolism in short-term experimental diabetic retinopathy. Diabetes. 815-820.
[44] MacMicking J, X. Q., Nathan C. (1997) Nitric oxide and macrophage function. AnnuRev Immunol. 323-350.
[45] Meyer P, C. C., Schlotzer-Schrehardt U, Flammer J, Haefliger 10 (1999) Localization of nitric oxide synthase isoforms in porcine ocular tissues. Curr Eye Res. 375-380.
[46] Miller, J. W., Adamis, A. P., Shima, D. T., D'Amore, P. A., Moulton, R. S., O'Reilly, M. S., Folkman, J., Dvorak, H. F., Brown, L. F., Berse, B., Yeo, T.-K., Yeo, K.-T (1994) Vascular endothelial growth factor/vascular permeability factor is temporally and spatially correlated with ocular angiogenesis in a primate model. Am. J. Pathol. 574-584.
[47] Moncada S, P. R., Higgs E A (1991) Nitric oxide: physiology, pathophysiology, and pharmacology. Pharmacol Rev. 109-142.
[48] Moss S E, K. R., Klein B E K (1998) The 14-year incidence of visual loss in a diabetic population. Ophthalmology. 998-1003.
[49] Murohara T, A. T., Silver M, Bauters C, Masuda H, Kalka C, Kearney M, Chen D, Symes J F, Fishman M C, Huang P L, Isner J M (1998) Nitric oxide synthase modulates angiogenesis response to tissue ischemia. J Clin Invest. 2567-2578.
[50] Murphy S, S. M., Agullo L, Garcia A, Feinstein D L, Galea E, Reis D J, Minc-Golomb D, Schwartz J P (1993) Synthesis of nitric oxide in CNS glial cells. Trends Neurosci. 323-328.
[51] Ogata N, Y. R., Yamamoto C, Miyashiro M, Kimoto T, Takahashi K, Maruyama K, Uyama M (1998) Expression of vascular endothelial growth factor and its receptor, KDR, following retinal-ischemia-reperfusion injury in the rat. Curr Eye Res. 1087-1096.
[52] Ozaki, H., Seo, M.-S., Ozaki, K., Yamada, H., Yamada, E., Okamoto, N., Hofmann, F., Wood, J. M., Campochiaro, P. A (2000) Blockade of vascular endothelial cell growth factor receptor signaling is sufficient to completely prevent retinal neovascularization. Am. J. Pathol. 697-707.
[53] Ozaki H, Y. A., Della N, Ozaki K, Luna J D, Yamada H, Hackett S F, Okamoto N, Zack D J, Semenza G L, Campochiaro P A (1999) Hypoxia inducible factor-1alpha is increased in ischemic retina: temporal and spatial correlation with VEGF expression. Invest Ophthalmol V is Sci. 182-189.
[54] Pekny M, L. P., Pekna M, Eliasson C, Berthold C H, Westermark B, Betsholtz C (1995) Mice lacking glial fibrillary acidic protein display astrocytes devoid of intermediate filaments but develop and reproduce normally. EMBO J. 1590-1598.
[55] Pekny M, N. M. (2005) Astrocyte activation and reactive gliosis. Glia. 427-434. [56] Phillips, G. D., Stone, A. M (1994) PDGF-BB induced chemotaxis is impaired in aged capillary endothelial cells. Mech. Ageing Dev. 189-196.
[57] Reichenbach A, F. F., Enzmann V, Bringmann A, Pannicke T, Francke M, Biedermann B, Kuhrt H, Stolzenburg J U, Skatchkov S N, Heinemann U, Wiedemann P, Reichelt W (1997) The Müller (glial) cell in normal and diseased retina: a case for single-cell electrophysiology. Ophthalmic Res. 326-340.
[58] Runnger-Brandle E, D. A., Leuenberger P M (2000) Glial reactivity, an early feature of diabetic Tetinopathy. Invest Ophthalmol V is Sci. 1971-1980.
[59] Russ P K, G. G. H. F. (2001) Retinal vascular permeability determined by dual-tracer fluorescence angiography. Annals of Biomedical Engineering. 638-647.
[60] Rutka J T, S. S. (1993) Transfection of human astrocytoma cells with glial fibrillary acidic protein complementary DNA: analysis of expression, proliferation, and tumorigenicity. Cancer Res. 3624-3631.
[61] Sahel J A, A. D., Lessell S (1990) Proliferation of retinal glia and excitatory amino acids. Ophtalmologie. 13-16.
[62] Salhia B, A. L., Roncari L, Wu X, Shannon P, Guha A (2000) Expression of vascular endothelial growth factor by reactive astrocytes and associated neoangiogenesis. Brain Res. 87-97.
[63] Sennlaub F, C. Y., Goureau O (2001) Inducible nitric oxide synthase mediates the change from retinal to vitreal neovascularization in ischemic retinopathy. J Clin Invest. 107: 717-725.
[64] Seo M S, K. N., Ozaki H, et al (1999) Dramatic inhibition of retinal and choroidal neovascularization by oral administration of a kinase inhibitor. Am J. Pathol. 1743-1753.
[65] Seo M S, O, N., Vinores M A, Vinores S A, Hackett S F, Yamada H, Yamada E, Derevjanik N L, LaRochelle W, Zack D J, Campochiaro P A (2000) Photoreceptor-specific expression of platelet-derived growth factor-B results in traction retinal detachment. Am J. Pathol. 995-1005.
[66] Shizukuda Y, T. S., Yokota R, Ware J A (1999) Vascular endothelial growth factorinduced endothelial cell migration and proliferation depend on a nitric oxide-mediated decrease in protein kinase C activity. Circ Res. 247-256.
[67] Smith, L. E. H., Wesolowski, E., McLellan, A., Kostyk, S. K., D'Amato, R., Sullivan, R., D'Amore, P. A (1994) Invest. Ophthalmol. Vis. Sci. Oxygen-induced retinopathy in the mouse. 101-111.
[68] Stitt A W, B. T., McMullen C B, Gardiner T A, Archer D B (2000) Advanced glycation end products induce blood-retinal barrier dysfunction in normoglycemic rats. Mol Cell Biol Res Commun. 380-388.
[69] Tackenberg M A, T. B., Swift J S, Jiang C, Redenti S, Greenberg K P, Flannery J G, Reichenbach A, Young M J (2009) Müller cell activation, proliferation and migration following laser injury. Mol. Vis. 2009 Sep. 17; 15:1886-96.: 1886-1896.
[70] Takamiya Y, K. S., Toya S, Otani M, Mikoshiba K, Tsukada Y. (1986) Possible association of platelet-derived growth factor (PDGF) with the appearance of reactive astrocytes following brain injury in situ. Brain Res. 305-309.
[71] Watanabe T, R. M. (1988) Retinal astrocytes are immigrants from the optic nerve. Nature. 834-837.
[72] Witzenbichler B, A. T., Murohara T, Silver M, Spyridopoulos I, Magner M, Principe N, Kearney M, Hu J S, Isner J M. (1998) Vascular endothelial growth factor-C (VEGFC/VEGF-2) promotes angiogenesis in the setting of tissue ischemia. Am J. Pathol. 381-394.
[73] Yasuda Y, T. N., Shimoda T, Satoh S, Ogitani E, Fujita S (2004) Relationship between S100beta and GFAP expression in astrocytes during infarction and glial scar formation after mild transient ischemia. Brain Res. 20-31.
[74] Yu A C H, L. Y., Eng L F (1991) Inhibition of GFAP synthesis by antisense RNA in astrocytes. J Neurosci Res. 72-79.
[75] Yu A C H, L. Y., Eng L F (1993) Astrogliosis in culture:I The model and the effect of antisense oligonucleotides on glial fibrillary acidic protein synthesis. J Neurosci Res. 295-303.
[76] Yu X, X. Z., Mi M, Xu H, Zhu J, Wei N, Chen K, Zhang Q, Zeng K, Wang J, Chen F, Tang Y. (2008) Dietary taurine supplementation ameliorates diabetic retinopathy via antiexcitotoxicity of glutamate in streptozotocin-induced Sprague-Dawley rats. Neurochem Res. 500-507.

1-19. (canceled)

20. A method for treating an acute neuronal disorder, and/or for the treatment, reversal or attenuation of reactive gliosis and reactive gliosis directly or indirectly associated with an eye disease, disease of the retina, or retinal disorder; comprising administering to a subject in need thereof an effective amount of a therapeutic agent comprising a pyrazolyl compound comprising the chemical structure of formula I 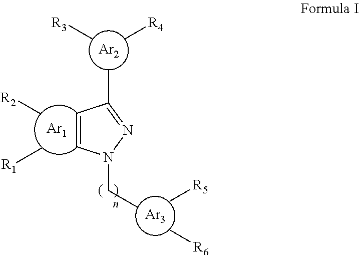
wherein
Ar₁ forms an aromatic or heteroaromatic ring, or is phenyl, wherein the aromatic or heteroaromatic ring is optionally substituted;
Ar₂ is selected from furyl, phenyl, alkyl, aryl, or heterocyclyl, wherein the alkyl, aryl, or heterocyclyl is optionally substituted;
Ar₃ is phenyl or substituted phenyl; n is 1 to 10;
or an enantiomer, racemic form or mixture, prodrug, or analog of the compound, or comprising administering to a subject in need thereof a pharmaceutical composition comprising such compound.

21. The method of claim 20, wherein
each of Ar₁, and Ar₃ is phenyl;
Ar₂ is furyl or phenyl;
one of R₁ and R₂ is H, and the other is H, C₁-C₆ alkyl or halogen;
one of R₃ and R₄ is H, and the other is CH₂OH;
each of R₅ and R₆ is H; n is 1 to 10
or an enantiomer, racemic form or mixture, prodrug, or analog of the compound.

22. The method of claim 20, wherein Ar₂ is 5′-furyl.

23. The method of claim 20, wherein R₃ is substituted at position 2 of furyl and wherein R₃ is CH₂OH and R₄ is H.

24. The method of claim 20, wherein R₁ and R₂ are substituted at positions 4 and 5 of phenyl and wherein R₁ is H, and R₂ is CH₃.

25. The method of claim 20, wherein R₁ is H, and R₂ is F.

26. The method of claim 20, wherein said compound is 3-(5′-hydroxymethyl-2′-furyl)-1-benzylindazole (YC-1) and has the chemical structure of Formula II: 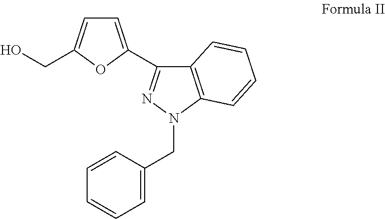 [SMILES: OCc1ccc(-c2nn(Cc3ccccc3)c3ccccc23)o1]
or an enantiomer, racemic form or mixture, prodrug, or analog of the compound.

27. The method of claim 20, wherein said compound is 1-benzyl-3-(5-methyl-furan-2-yl)-1H-indazole, 1-benzyl-3-(5-methoxymethyl-furan-2-yl)-1H-indazole, or 1-benzyl-3-(5′-methoxymethyl-2′-furyl)-indazole, or an enantiomer, racemic form or mixture, prodrug, or analog of the compound.

28. The method of claim 20 for use in reducing PDGF-B, GFAP and/or iNOS expression and protein levels, in vivo or in vitro; reduction of filopodial length and number in endothelial tip cells; inhibition of ischemic pathologic neovascular response; promotion of physiological retinal microvascular repair and/or intra-retinal revascularization of the avascular retina.

29. The method of claim 20, for the treatment, reversal or the attenuation of gliosis, astrogliosis, or reactive gliosis, directly or indirectly associated with a retinal or eye disease.

30. The method of claim 20 for the treatment, reversal or the attenuation of reactive gliosis associated with diabetic retinopathy or retinal neovascularization.

31. The method according to claim 20, wherein the pharmaceutical composition, comprises the compound as defined in claim 20 and a pharmaceutically acceptable carrier.

32. The method of claim 31, used to treat a disease or disorder associated with retinopathy, ischemic retinopathy, hypertensive retinopathy, retinal neovascularization, macular degeneration, Meckel syndrome, autosomal recessive inheritance diseases, Bardet-Biedl syndrome, retinal vessel occlusions and retinal artery occlusion, cytomegalovirus (CMV) retinitis, diabetic retinopathy, diabetic eye problems, epi-retinal membrane, flashing lights, eye floaters, flashes and posterior vitreous detachments, macular edema (CME), macular holes, macular translocation, cancers affecting the retina, melanoma, retinoblastoma (PDQ), retinal tear and retinal detachment, retinal detachment repair, proliferative vitreoretinopathy (PVR), photodynamic therapy (PDT), subretinal neovascular membranes and surgery, vitrectomy, usher syndrome, retinoschisis, retinitis pigmentosa (RP), retinal tear, Bietti's crystalline dystrophy, choroideremia, retinopathy of prematurity (ROP), Behcet's disease, central serous choroidopathy, amaurosis fugax, Leber congenital amaurosis, juvenile retinoschisis, refsum disease, neuropathy, ataxia, retinitis pigmentosa, familial exudative vitreoretinopathy, and/or choroideremia.

33. The method of claim 31, wherein said method is for the treatment, reversal or the attenuation of reactive gliosis associated with diabetic retinopathy or retinal neovascularization.

34. The method of claim 31 wherein the pharmaceutical composition is in a form selected from tablets, lozenges, pills, dragees, capsules, liquids, gels, syrups, slurries, suspensions, solutions and emulsions.

35. The method of claim 31, wherein said pharmaceutical composition is for oral, rectal, transmucosal, transdermal, intestinal, parenteral, intramuscular, intrathecal, direct intraventricular, intravenous, intraperitoneal, intranasal, intraocular, or subcutaneous administration.

36. The method according to claim 20, wherein the compound is 3-(5′-hydroxymethyl-2′-furyl)-1-benzylindazole and the disease or disorder is reactive gliosis.

37. The method, according to claim 36, wherein the disease or disorder is associated with an eye disease.
